(19) United States
(12) Patent Application Publication
(10) Pub. No.: US 2005/0059035 A1
(43) Pub. Date: Mar. 17, 2005

(54) Methods and compositions for the amplification of mutations in the diagnosis of cystic fibrosis

(75) Inventors: Doug Hui Huang, Irvine, CA (US); Feras Hantash, Dana Point, CA (US); Ron Kagan, Mission Viejo, CA (US)

Correspondence Address: FOLEY & LARDNER, P.O. BOX 80278, SAN DIEGO, CA, 92138-0278, US

(73) Assignee: Quest Diagnostics Incorporated

(21) Appl. No.: 10/775,650

(22) Filed: Feb. 9, 2004

Related U.S. Application Data
(63) Continuation-in-part of application No. 10/659,582, filed on Sep. 9, 2003.

(51) Int. Cl. C12Q 1/68; C07H 21/04; C12P 19/34

(52) U.S. Cl. 435/6; 435/91.2; 536/23.2

(57) ABSTRACT

The present invention provides nucleic acid primers for amplifying DNA sequences of normal and mutant cystic fibrosis (CF) genes. These primers enable the construction of assays that use the amplified CF genes to detect of the presence of normal or mutant CF sequence, thereby enabling the detection of the genotype of cystic fibrosis in a biological sample. Various pairs of primers suitable for amplifying different CFTR gene segments are provided which are suitable for use in a multiplex amplification format.

This application is a continuation-in-part of U.S. application Ser. No. 10/659,582, filed Sep. 9, 2003, the entire contents of which are incorporated by reference herein for all purposes.

### FIELD OF THE INVENTION

The present invention relates to nucleotide sequences useful as primers for amplifying portions of the cystic fibrosis transmembrane regulator (CFTR) gene where cystic fibrosis (CF) mutations are known to arise, and use of the amplified sequence to identify the presence or absence of CF mutant sequences in a biological sample.

### BACKGROUND OF THE INVENTION

The following description of the background of the invention is provided simply as an aid in understanding the invention and is not admitted to describe or constitute prior art to the invention.

Cystic fibrosis (CF) is the most common severe autosomal recessive genetic disorder in the Caucasian population. It affects approximately 1 in 2,500 live births in North America (Boat et al, The Metabolic Basis of Inherited Disease, 6th ed, pp 2649-2680, McGraw Hill, NY (1989)). Approximately 1 in 25 persons are carriers of the disease. The responsible gene has been localized to a 250,000 base pair genomic sequence present on the long arm of chromosome 7. This sequence encodes a membrane-associated protein called the “cystic fibrosis transmembrane regulator” (or “CFTR”). There are greater than 1000 different mutations in the CFTR gene, having varying frequencies of occurrence in the population, presently reported to the Cystic Fibrosis Genetic Analysis Consortium. These mutations exist in both the coding regions (e.g., ΔF508, a mutation found on about 70% of CF alleles, represents a deletion of a phenylalanine at residue 508) and the non-coding regions (e.g., the 5T, 7T, and 9T mutations correspond to a sequence of 5, 7, or 9 thymidine bases located at the splice branch/acceptor site of intron 8) of the CFTR gene.

The major symptoms of cystic fibrosis include chronic pulmonary disease, pancreatic exocrine insufficiency, and elevated sweat electrolyte levels. The symptoms are consistent with cystic fibrosis being an exocrine disorder. Although recent advances have been made in the analysis of ion transport across the apical membrane of the epithelium of CF patient cells, it is not clear that the abnormal regulation of chloride channels represents the primary defect in the disease.

### SUMMARY OF THE INVENTION

The present invention provides compositions and methods for amplifying CFTR nucleic acid sequences and for using such amplified sequence to identify the presence of absence of CF mutations in the CFTR gene. In particular, nucleic acid primers are provided herein for amplifying segments of the CFTR gene that are known to contain mutant cystic fibrosis (CF) nucleic acid sequence. These primers therefore enable the construction of assays that utilize amplification methods, preferably the polymerase chain reaction (PCR), to amplify the nucleic acid sequences in a biological sample for detection of mutant gene sequence. The present invention therefore further discloses methods for detecting individual mutant CF sequence in the amplified product(s).

In a first aspect, the present invention provides one or more substantially pure nucleic acid sequences, and/or complementary sequences thereof, that can be used as primers to amplify segments of the CFTR gene where CF mutant nucleic acid sequences are known to arise.

The primers of the present invention hybridize to a CFTR coding sequence or a CFTR non-coding sequence, or to a complement thereof. Suitable primers are capable of hybridizing to coding or non-coding CFTR sequence under stringent conditions. The primers may be complementary to CF predetermined nucleic acid sequences that are associated with cystic fibrosis or may flank one or more such sequences. Preferred primers are those that flank mutant CF sequences. Primers may be labeled with any of a variety of detectable agents such as radioisotopes, dyes, fluorescent molecules, haptens or ligands (e.g., biotin), and the like. In a preferred approach, the primer are labeled with biotin. The biotin label is preferably attached to the 5′ end of the primer.

By “predetermined sequence” is meant a nucleic acid sequence that is known to be associated with cystic fibrosis. Predetermined sequence that is known to be associated with cystic fibrosis includes mutant CF nucleotide sequence.

By “mutant CF nucleic acid sequence,” “CF mutant sequences,” or “genotype for cystic fibrosis” is meant one or more CFTR nucleic acid sequences that are associated or correlated with cystic fibrosis. These mutant CF sequences may be correlated with a carrier state, or with a person afflicted with CF. The nucleic acid sequences are preferably DNA sequences, and are preferably genomic DNA sequences; however, RNA sequences such as mRNA or hnRNA may also contain nucleic acid sequences that are associated with cystic fibrosis. Mutations in the cystic fibrosis gene are described, for example, in U.S. Pat. No. 5,981,178 to Tsui et al., including mutations in the cystic fibrosis gene at amino acid positions 85, 148, 178, 455, 493, 507, 542, 549, 551, 560, 563, 574, 1077, and 1092, among others. Also disclosed are mutant DNA at nucleotide sequence positions, 621+1, 711+1, 1717−1 and 3659, which encode mutant CFTR (Cystic Fibrosis Transmembrane Conductance Regulator) polypeptide. Preferred sequences known to be associated with CF are described hereinafter, e.g., in Table 1.

By “carrier state” is meant a person who contains one CFTR allele that is a mutant CF nucleic acid sequence, but a second allele that is not a mutant CF nucleic acid sequence. CF is an “autosomal recessive” disease, meaning that a mutation produces little or no phenotypic effect when present in a heterozygous condition with a non-disease related allele, but produces a “disease state” when a person is homozygous, i.e., both CFTR alleles are mutant CF nucleic acid sequences.

By “primer” is meant a sequence of nucleic acid, preferably DNA, that hybridizes to a substantially complementary target sequence and is recognized by DNA polymerase to begin DNA replication.

By “substantially complementary” is meant that two sequences hybridize under stringent hybridization conditions. The skilled artisan will understand that substantially complementary sequences need not hybridize along their entire length. In particular, substantially complementary sequences comprise a contiguous sequence of bases that do not hybridize to a target sequence, positioned 3′ or 5′ to a contiguous sequence of bases that hybridize under stringent hybridization conditions to a target sequence.

By “flanking” is meant that a primer hybridizes to a target nucleic acid adjoining a region of interest sought to be amplified on the target. The skilled artisan will understand that preferred primers are pairs of primers that hybridize 3′ from a region of interest, one on each strand of a target double stranded DNA molecule, such that nucleotides may be add to the 3′ end of the primer by a suitable DNA polymerase. Primers that flank mutant CF sequences do not actually anneal to the mutant sequence but rather anneal to sequence that adjoins the mutant sequence.

By “isolated” a nucleic acid (e.g., an RNA, DNA or a mixed polymer) is one which is substantially separated from other cellular components which naturally accompany such nucleic acid. The term embraces a nucleic acid sequence which has been removed from its naturally occurring environment, and includes recombinant or cloned DNA isolates, oligonucleotides, and chemically synthesized analogs or analogs biologically synthesized by heterologous systems.

By “substantially pure” a nucleic acid, represents more than 50% of the nucleic acid in a sample. The nucleic acid sample may exist in solution or as a dry preparation.

By “complement” is meant the complementary sequence to a nucleic acid according to standard Watson/Crick pairing rules. For example, a sequence (SEQ ID NO: 1) 5′-GCGGTCCCAAAAG-3′ has the complement (SEQ ID NO: 2) 5′-CTTTTGGGACCGC-3′. A complement sequence can also be a sequence of RNA complementary to the DNA sequence or its complement sequence, and can also be a cDNA.

By “coding sequence” is meant a sequence of a nucleic acid or its complement, or a part thereof, that can be transcribed and/or translated to produce the mRNA for and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the encoding sequence can be deduced therefrom.

By “non-coding sequence” is meant a sequence of a nucleic acid or its complement, or a part thereof, that is not transcribed into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, etc.

In preferred embodiments the substantially pure nucleic acid sequence(s) is(are) a DNA (or RNA equivalent) that is any of the following:

| | |
|---|---|
| 5′- GCGGTCCCAAAAGGGTCAGTTGTAGGAAGT | SEQ ID NO: 3 |
| CACCAAAG -3′ (g4e1F) | |
| 5′- GCGGTCCCAAAAGGGTCAGTCGATACAGAA | SEQ ID NO: 4 |
| TATATGTGCC -3′ (g4e2R) | |
| 5′- GCGGTCCCAAAAGGGTCAGTGAATCATTCA | SEQ ID NO: 5 |
| GTGGGTATAAGCAG -3′ (g19i2F) | |
| 5′- GCGGTCCCAAAAGGGTCAGTCTTCAATGCA | SEQ ID NO: 6 |
| CCTCCTCCC -3′ (q19i3R) | |
| 5′- GCGGTCCCAAAAGGGTCAGTAGATACTTCA | SEQ ID NO: 7 |
| ATAGCTCAGCC -3′ (g7e1F) | |
| 5′- GCGGTCCCAAAAGGGTCAGTGGTACATTAC | SEQ ID NO: 8 |
| CTGTATTTTGTTT -3′ (g7e2R) | |
| 5′- GCGGTCCCAAAAGGGTCAGTGTGAATCGAT | SEQ ID NO: 9 |
| GTGGTGACCA-3′ (s12e1F) | |
| 5′- GCGGTCCCAAAAGGGTCAGTCTGGTTTAGC | SEQ ID NO: 10 |
| ATGAGGCGGT -3′ (s12e1R) | |
| 5′- GCGGTCCCAAAAGGGTCAGTTTGGTTGTGC | SEQ ID NO: 11 |
| TGTGGCTCCT -3′ (g14be1F) | |
| 5′- GCGGTCCCAAAAGGGTCAGTACAATACATA | SEQ ID NO: 12 |
| CAAACATAGTGG -3′ (g14be2R) | |
| 5′- GCGGTCCCAAAAGGGTCAGTGAAAGTATTT | SEQ ID NO: 13 |
| ATTTTTTCTGGAAC -3′ (q21e1F) | |
| 5′- GCGGTCCCAAAAGGGTCAGTGTGTGTAGAA | SEQ ID NO: 14 |
| TGATGTCAGCTAT -3′ (q21e2R) | |
| 5′- GCGGTCCCAAAAGGGTCAGTCAGATTGAGC | SEQ ID NO: 15 |
| ATACTAAAAGTG-3′ (g11e1F) | |
| 5′- GCGGTCCCAAAAGGGTCAGTTACATGAATG | SEQ ID NO: 16 |
| ACATTTACAGCA -3′ (g11e2R) | |
| 5′- GCGGTCCCAAAAGGGTCAGTAAGAACTGGA | SEQ ID NO: 17 |
| TCAGGGAAGA -3′ (g20e1F) | |
| 5′- GCGGTCCCAAAAGGGTCAGTTCCTTTTGCT | SEQ ID NO: 18 |
| CACCTGTGGT -3′ (g20e2R) | |
| 5′- GCGGTCCCAAAAGGGTCAGTGGTCCCACTT | SEQ ID NO: 19 |
| TTTATTCTTTTGC -3′ (q3e2F) | |
| 5′- GCGGTCCCAAAAGGGTCAGTTGGTTTCTTA | SEQ ID NO: 20 |
| GTGTTTGGAGTTG -3′ (q3e2R) | |
| 5′- GCGGTCCCAAAAGGGTCAGTTGGATCATGG | SEQ ID NO: 21 |
| GCCATGTGC -3′ (g9e9F) | |
| 5′- GCGGTCCCAAAAGGGTCAGTACTACCTTGC | SEQ ID NO: 22 |
| CTGCTCCAGTGG -3′ (g9e9R) | |
| 5′- GCGGTCCCAAAAGGGTCAGTAGGTAGCAGC | SEQ ID NO: 23 |
| TATTTTTATGG -3′ (g13e2F) | |
| 5′- GCGGTCCCAAAAGGGTCAGTTAAGGGAGTC | SEQ ID NO: 24 |
| TTTTGCACAA -3′ (g13e2R) | |
| 5′- GCGGTCCCAAAAGGGTCAGTGCAATTTTGG | SEQ ID NO: 25 |
| ATGACCTTC -3′ (q16i1F) | |
| 5′- GCGGTCCCAAAAGGGTCAGTTAGACAGGAC | SEQ ID NO: 26 |
| TTCAACCCTC -3′ (q16i2R) | |
| 5′- GCGGTCCCAAAAGGGTCAGTGGTGATTATG | SEQ ID NO: 27 |
| GGAGAACTGG -3′ (q10e10F) | |
| 5′- GCGGTCCCAAAAGGGTCAGTATGCTTTGAT | SEQ ID NO: 28 |
| GACGCTTC -3′ (q10e11R) | |
| 5′- GCGGTCCCAAAAGGGTCAGTTTCATTGAAA | SEQ ID NO: 29 |
| AGCCCGAC -3′ (q19e12F) | |
| 5′- GCGGTCCCAAAAGGGTCAGTCACCTTCTGT | SEQ ID NO: 30 |
| GTATTTTGCTG -3′ (q19e13R) | |
| 5′- GCGGTCCCAAAAGGGTCAGTAAGTATTGGA | SEQ ID NO: 31 |
| CAACTTGTTAGTCTC-3′ (q5e12F) | |
| 5′- GCGGTCCCAAAAGGGTCAGTCGCCTTTCCA | SEQ ID NO: 32 |
| GTTGTATAATTT -3′ (q5e13R) | |

or a complement of one or more of these sequences.

In another aspect, the present invention provides methods of amplifying CF nucleic acids to determine the presence of one or more mutant CF sequences. In accordance with this method, nucleic acid suspected of containing mutant CF sequences are amplified using one or more primers that flank one or more predetermined nucleic acid sequences that are associated with cystic fibrosis under conditions such that the primers will amplify the predetermined nucleic acid sequences, if present. In preferred embodiments, the amplification primers used are one or more of the sequences designated as SEQ ID NO: 3 through SEQ ID NO: 32, or a complement of one or more of these sequences. In preferred embodiments, pairs of primers are used for amplification, the pairs being SEQ ID NOs: 3 and 4, 5 and 6, 7 and 8, 9 and 10, 11 and 12, 13 and 14, 15 and 16, 17 and 18, 19 and 20, 21 and 22, 23 and 24, 25 and 26, 27 and 28, 29 and 30, and 31 and 32. In further preferred embodiments, the number of pairs of primers is 5 pairs of primers, even more preferably 10 pairs of primers and most preferably 15 pairs of primers.

In the case where the 15 pairs of primers are used in combinations, primer sets are added in the following ratios determined as the moles (mole is defined as mass/molecular weight of a compound) of primers for exon 12 and 21 (SEQ ID NO: 9, 10, 13 and 14) relative to the moles of each other primer sets, the ratio being about 2 for exons 4 and i19 (SEQ ID NOs; 3-6), about 3.2 for exons 19, 7, 11 and i5 (SEQ ID NOs; 7, 8, 15, 16, and 29-32), about 4 for exons 3 and 14 (SEQ ID NOs; 11, 12, 19, 20), about 4.8 for exons 16, 20, 13 and 10 (SEQ ID NOs; 17, 18, 23 and 28), and about 8 for exon 9 (SEQ ID NOs; 22 and 21). Thus, the amount of exon 12 and 21 primers added is about (SEQ ID NO: 9, 10, 13 and 14) 2 fold that of exons 4 and i19 (SEQ ID NOs; 3-6), about 3.2 fold that of exons 19, 7 and i5 (SEQ ID NOs; 7, 8, 15, 16, and 29-32), about 4 fold that of exons 3 and 14 (SEQ ID NOs; 11, 12, 19, 20), about 4.8 fold that of exons 16, 20, 13 and 10 (SEQ ID NOs; 17, 18, 23 and 28), and about 8 fold that of exon 9 (SEQ ID NOs; 22 and 21).

The method of identifying the presence or absence of mutant CF sequence by amplification can be used to determine whether a subject has a genotype containing one or more nucleotide sequences correlated with cystic fibrosis. The presence of a wildtype or mutant sequence at each predetermined location can be ascertained by the invention methods.

By “amplification” is meant one or more methods known in the art for copying a target nucleic acid, thereby increasing the number of copies of a selected nucleic acid sequence. Amplification may be exponential or linear. A target nucleic acid may be either DNA or RNA. The sequences amplified in this manner form an “amplicon.” While the exemplary methods described hereinafter relate to amplification using the polymerase chain reaction (“PCR”), numerous other methods are known in the art for amplification of nucleic acids (e.g., isothermal methods, rolling circle methods, etc.). The skilled artisan will understand that these other methods may be used either in place of, or together with, PCR methods.

The nucleic acid suspected of containing mutant CF sequence may be obtained from a biological sample. By “biological sample” is meant a sample obtained from a biological source. A biological sample can, by way of non-limiting example, consist of or comprise blood, sera, urine, feces, epidermal sample, skin sample, cheek swab, sperm, amniotic fluid, cultured cells, bone marrow sample and/or chorionic villi. Convenient biological samples may be obtained by, for example, scraping cells from the surface of the buccal cavity. The term biological sample includes samples which have been processed to release or otherwise make available a nucleic acid for detection as described herein. For example, a biological sample may include a cDNA that has been obtained by reverse transcription of RNA from cells in a biological sample.

By “subject” is meant a human or any other animal which contains as CFTR gene that can be amplified using the primers and methods described herein. A subject can be a patient, which refers to a human presenting to a medical provider for diagnosis or treatment of a disease. A human includes pre and post natal forms. Particularly preferred subjects are humans being tested for the existence of a CF carrier state or disease state.

By “identifying” with respect to an amplified sample is meant that the presence or absence of a particular nucleic acid amplification product is detected. Numerous methods for detecting the results of a nucleic acid amplification method are known to those of skill in the art.

In another aspect the present invention provides kits for one of the methods described herein. In various embodiments, the kits contain one or more of the following components in an amount sufficient to perform a method on at least one sample: one or more primers of the present invention, one or more devices for performing the assay, which may include one or more probes that hybridize to a mutant CF nucleic acid sequence, and optionally contain buffers, enzymes, and reagents for performing a method of detecting a genotype of cystic fibrosis in a nucleic acid sample.

The following description of the background of the invention is provided simply as an aid in understanding the invention and is not admitted to describe or constitute prior art to the invention.

### BRIEF DESCRIPTION OF THE FIGURES

FIG. 1 is a table showing the designations of biotinylated primers and their nucleotide sequence for use in the detection of mutant CF genotype. Primers numbered from 1-30 relate to SEQ ID NOs. 3-32, respectively.

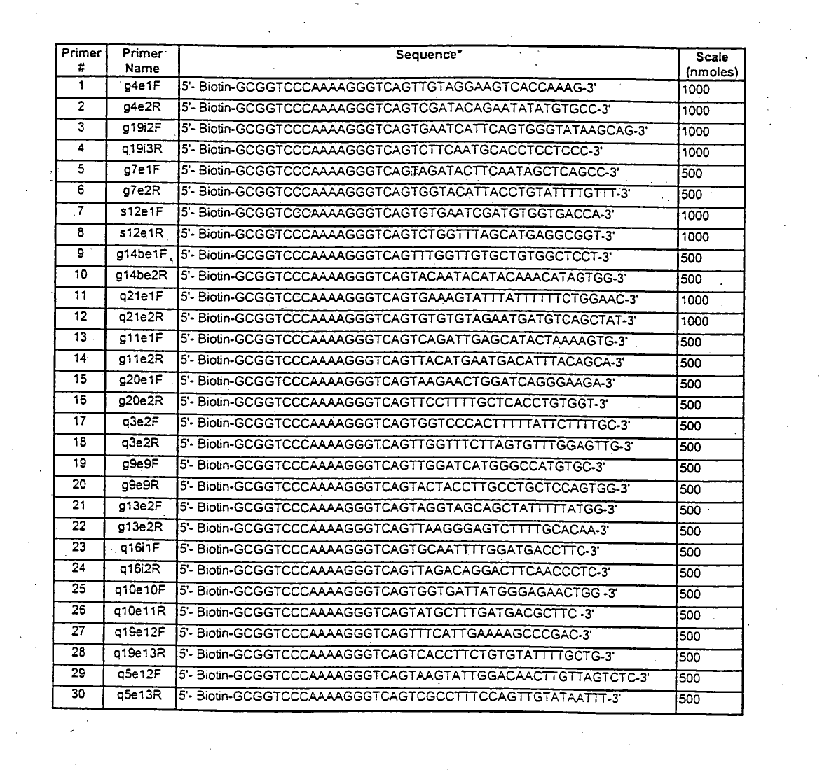

### DETAILED DESCRIPTION OF THE INVENTION

The present invention provides specific primers that aid in the detection of mutant CF genotype. Such primers enable the amplification of segments of the CFTR gene that are known to contain mutant CF sequence from a nucleic acid containing biological sample. By amplifying specific regions of the CFTR gene, the invention primers facilitate the identification of wildtype or mutant CF sequence at a particular location of the CFTR gene. Accordingly, there is provided a substantially purified nucleic acid sample comprising one or more nucleic acids having sequences selected from the group consisting of:

| | |
|---|---|
| 5′- GCGGTCCCAAAAGGGTCAGTTGTAGGAAGT | (SEQ ID NO: 3) |
| CACCAAAG -3′, | |
| 5′- GCGGTCCCAAAAGGGTCAGTCGATACAGAA | (SEQ ID NO: 4) |
| TATATGTGCC -3′, | |
| 5′- GCGGTCCCAAAAGGGTCAGTGAATCATTCA | (SEQ ID NO: 5) |
| GTGGGTATAAGCAG -3′, | |
| 5′- GCGGTCCCAAAAGGGTCAGTCTTCAATGCA | (SEQ ID NO: 6) |
| CCTCCTCCC -3′, | |
| 5′- GCGGTCCCAAAAGGGTCAGTAGATACTTCA | (SEQ ID NO: 7) |
| ATAGCTCAGCC -3′, | |
| 5′- GCGGTCCCAAAAGGGTCAGTGGTACATTAC | (SEQ ID NO: 8) |
| CTGTATTTTGTTT -3′, | |
| 5′- GCGGTCCCAAAAGGGTCAGTGTGAATCGAT | (SEQ ID NO: 9) |
| GTGGTGACCA -3′, | |
| 5′- GCGGTCCCAAAAGGGTCAGTCTGGTTTAGC | (SEQ ID NO: 10) |
| ATGAGGCGGT -3′, | |
| 5′- GCGGTCCCAAAAGGGTCAGTTTGGTTGTGC | (SEQ ID NO: 11) |
| TGTGGCTCCT -3′, | |
| 5′- GCGGTCCCAAAAGGGTCAGTACAATACATA | (SEQ ID NO: 12) |
| CAAACATAGTGG -3′, | |
| 5′- GCGGTCCCAAAAGGGTCAGTGAAAGTATTT | (SEQ ID NO: 13) |
| ATTTTTTCTGGAAC -3′, | |
| 5′- GCGGTCCCAAAAGGGTCAGTGTGTGTAGAA | (SEQ ID NO: 14) |
| TGATGTCAGCTAT -3′, | |
| 5′- GCGGTCCCAAAAGGGTCAGTCAGATTGAGC | (SEQ ID NO: 15) |
| ATACTAAAAGTG -3′, | |
| 5′- GCGGTCCCAAAAGGGTCAGTTACATGAATG | (SEQ ID NO: 16) |
| ACATTTACAGCA -3′, | |
| 5′- GCGGTCCCAAAAGGGTCAGTAAGAACTGGA | (SEQ ID NO: 17) |
| TCAGGGAAGA -3′, | |
| 5′- GCGGTCCCAAAAGGGTCAGTTCCTTTTGCT | (SEQ ID NO: 18) |
| CACCTGTGGT -3′, | |
| 5′- GCGGTCCCAAAAGGGTCAGTGGTCCCACTT | (SEQ ID NO: 19) |
| TTTATTCTTTTGC -3′, | |
| 5′- GCGGTCCCAAAAGGGTCAGTTGGTTTCTTA | (SEQ ID NO: 20) |
| GTGTTTGGAGTTG -3′, | |
| 5′- GCGGTCCCAAAAGGGTCAGTTGGATCATGG | (SEQ ID NO: 21) |
| GCCATGTGC -3′, | |
| 5′- GCGGTCCCAAAAGGGTCAGTACTACCTTGC | (SEQ ID NO: 22) |
| CTGCTCCAGTGG -3′, | |
| 5′- GCGGTCCCAAAAGGGTCAGTAGGTAGCAGC | (SEQ ID NO: 23) |
| TATTTTTATGG -3′, | |
| 5′- GCGGTCCCAAAAGGGTCAGTTAAGGGAGTC | (SEQ ID NO: 24) |
| TTTTGCACAA -3′, | |
| 5′- GCGGTCCCAAAAGGGTCAGTGCAATTTTGG | (SEQ ID NO: 25) |
| ATGACCTTC -3′, | |
| 5′- GCGGTCCCAAAAGGGTCAGTTAGACAGGAC | (SEQ ID NO: 26) |
| TTCAACCCTC -3′, | |
| 5′- GCGGTCCCAAAAGGGTCAGTGGTGATTATG | (SEQ ID NO: 27) |
| GGAGAACTGG -3′, | |
| 5′- GCGGTCCCAAAAGGGTCAGTATGCTTTGAT | (SEQ ID NO: 28) |
| GACGCTTC -3′, | |
| 5′- GCGGTCCCAAAAGGGTCAGTTTCATTGAAA | (SEQ ID NO: 29) |
| AGCCCGAC -3′, | |
| 5′- GCGGTCCCAAAAGGGTCAGTCACCTTCTGT | (SEQ ID NO: 30) |
| GTATTTTGCTG -3′, | |
| 5′- GCGGTCCCAAAAGGGTCAGTAAGTATTGGA | (SEQ ID NO: 31) |
| CAACTTGTTAGTCTC -3′, | |
| 5′- GCGGTCCCAAAAGGGTCAGTCGCCTTTCCA | (SEQ ID NO: 32) |
| GTTGTATAATTT -3′, | |

or a complementary nucleic acid sequence thereof.

The invention nucleic acids are useful for primer-directed amplification of CFTR gene segments known to contain CF mutations. The primers may be used individually or, more preferably in pairs that flank a particular CF gene sequence. Thus, SEQ ID NO: 3, 5′-GCGGTCCCAAAAGGGTCAGTTGTAGGAAGTCACCAAAG-3′ (g4e1F), and SEQ ID NO: 4, 5′-GCGGTCCCAAAAGGGTCAGTCGATACAGAATATATGTGCC-3′ (g4e2R), are preferably used together as forward (F) and reverse (R) primers; SEQ ID NO: 5, 5′-GCGGTCCCAAAAGGGTCAGTGAATCATTCAGTGGGTATAAGCAG-3′ (g19i2F), and SEQ ID NO: 6, 5′-GCGGTCCCAAAAGGGTCAGTCTTCAATGCACCTCCTCCC-3′ (q19i3R), are preferably used together as forward (F) and reverse (R) primers; SEQ ID NO: 7, 5′-GCGGTCCCAAAAGGGTCAGTAGATACTTCAATAGCTCAGCC-3′ (g7e1F), and SEQ ID NO: 8, 5′-GCGGTCCCAAAAGGGTCAGTGGTACATTACCTGTATTTTGTTT-3′ (g7e2R), are preferably used together as forward (F) and reverse (R) primers; SEQ ID NO: 9, 5′-GCGGTCCCAAAAGGGTCAGTGTGAATCGATGTGGTGACCA-3′ (s12e1F), and SEQ ID NO: 10, 5′-GCGGTCCCAAAAGGGTCAGTCTGGTTTAGCATGAGGCGGT-3′ (s12e1R), are preferably used together as forward (F) and reverse (R) primers; SEQ ID NO: 11, 5′-GCGGTCCCAAAAGGGTCAGTTTGGTTGTGCTGTGGCTCCT-3′ (g14be1F), and SEQ ID NO: 12, 5′-GCGGTCCCAAAAGGGTCAGTACAATACATACAAACATAGTGG-3′ (g14be2R), are preferably used together as forward (F) and reverse (R) primers; SEQ ID NO: 13, 5′-GCGGTCCCAAAAGGGTCAGTGAAAGTATTTATTTTTTCTGGAAC-3′ (q21e1F), and SEQ ID NO: 14 5′-GCGGTCCCAAAAGGGTCAGTGTGTGTAGAATGATGTCAGCTAT-3′ (q21e2R), are preferably used together as forward (F) and reverse (R) primers; SEQ ID NO: 15, 5′-GCGGTCCCAAAAGGGTCAGTCAGATTGAGCATACTAAAAGTG-3′ (g11e1F), and SEQ ID NO: 16, 5′-GCGGTCCCAAAAGGGTCAGTTACATGAATGACATTTACAGCA-3′ (g11e2R), are preferably used together as forward (F) and reverse (R) primers; SEQ ID NO: 17, 5′-GCGGTCCCAAAAGGGTCAGTAAGAACTGGATCAGGGAAGA-3′ (g20e1F), and SEQ ID NO: 18, 5′-GCGGTCCCAAAAGGGTCAGTTCCTTTTGCTCACCTGTGGT-3′ (g20e2R), are preferably used together as forward (F) and reverse (R) primers; SEQ ID NO: 19, 5′-GCGGTCCCAAAAGGGTCAGTGGTCCCACTTTTTATTCTTTTGC-3′ (q3e2F), and SEQ ID NO: 20 5′-GCGGTCCCAAAAGGGTCAGTTGGTTTCTTAGTGTTTGGAGTTG-3′ (q3e2R), are preferably used together as forward (F) and reverse (R) primers; SEQ ID NO: 21, 5′-GCGGTCCCAAAAGGGTCAGTTGGATCATGGGCCATGTGC-3′ (g9e9F), and SEQ ID NO: 22, 5′-GCGGTCCCAAAAGGGTCAGTACTACCTTGCCTGCTCCAGTGG-3′ (g9e9R), are preferably used together as forward (F) and reverse (R) primers; SEQ ID NO: 23, 5′-GCGGTCCCAAAAGGGTCAGTAGGTAGCAGCTATTTTTATGG-3′ (g13e2F), and SEQ ID NO: 24, 5′-GCGGTCCCAAAAGGGTCAGTTAAGGGAGTCTTTTGCACAA-3′ (g13e2R), are preferably used together as forward (F) and reverse (R) primers; SEQ ID NO: 25 5′-GCGGTCCCAAAAGGGTCAGTGCAATTTTGGATGACCTTC-3′ (q16i1F), and SEQ ID NO: 26 5′-GCGGTCCCAAAAGGGTCAGTTAGACAGGACTTCAACCCTC-3′ (q16i2R), are preferably used together as forward (F) and reverse (R) primers; SEQ ID NO: 27, 5′-GCGGTCCCAAAAGGGTCAGTGGTGATTATGGGAGAACTGG-3′ (q10e10F), and SEQ ID NO: 28, 5′-GCGGTCCCAAAAGGGTCAGTATGCTTTGATGACGCTTC-3′ (q10e11R), are preferably used together as forward (F) and reverse (R) primers; SEQ ID NO: 29, 5′-GCGGTCCCAAAAGGGTCAGTTTCATTGAAAAGCCCGAC-3′ (q19e12F), and SEQ ID NO: 30, 5′-GCGGTCCCAAAAGGGTCAGTCACCTTCTGTGTATTTTGCTG-3′ (q19e13R) are preferably used together as forward (F) and reverse (R) primers; and SEQ ID NO: 31, 5′-GCGGTCCCAAAAGGGTCAGTAAGTATTGGACAACTTGTTAGTCTC-3′ (q5e12F), and SEQ ID NO: 32, 5′-GCGGTCCCAAAAGGGTCAGTCGCCTTTCCAGTTGTATAATTT-3′ (q5e13R), are preferably used together as forward (F) and reverse (R) primers.

Accordingly, there is provided a method of amplifying a nucleic acid sequence, comprising, contacting a nucleic acid containing sample with reagents suitable for nucleic acid amplification including one or more pairs of primers flanking one or more predetermined nucleic acid sequences that are correlated with cystic fibrosis, and amplifying said one or more predetermined nucleic acid sequences, if present, wherein said primers are one or more pairs of nucleic acids selected from the group consisting of:

| | |
|---|---|
| 5′-GCGGTCCCAAAAGGGTCAGTTGTAGGAAGTCACCAAAG-3′, | (SEQ ID NO: 3) |
| 5′-GCGGTCCCAAAAGGGTCAGTCGATACAGAATATATGTGCC-3′, | (SEQ ID NO: 4) |
| 5′-GCGGTCCCAAAAGGGTCAGTGAATCATTCAGTGGGTATAAGCAG-3′, | (SEQ ID NO: 5) |
| 5′-GCGGTCCCAAAAGGGTCAGTCTTCAATGCACCTCCTCCC-3′, | (SEQ ID NO: 6) |
| 5′-GCGGTCCCAAAAGGGTCAGTAGATACTTCAATAGCTCAGCC-3′, | (SEQ ID NO: 7) |
| 5′-GCGGTCCCAAAAGGGTCAGTGGTACATTACCTGTATTTTGTTT-3′, | (SEQ ID NO: 8) |
| 5′-GCGGTCCCAAAAGGGTCAGTGTGAATCGATGTGGTGACCA-3′, | (SEQ ID NO: 9) |
| 5′-GCGGTCCCAAAAGGGTCAGTCTGGTTTAGCATGAGGCGGT-3′, | (SEQ ID NO: 10) |
| 5′-GCGGTCCCAAAAGGGTCAGTTTGGTTGTGCTGTGGCTCCT-3′, | (SEQ ID NO: 11) |
| 5′-GCGGTCCCAAAAGGGTCAGTACAATACATACAAACATAGTGG-3′, | (SEQ ID NO: 12) |
| 5′-GCGGTCCCAAAAGGGTCAGTGAAAGTATTTATTTTTTCTGGAAC-3′, | (SEQ ID NO: 13) |
| 5′-GCGGTCCCAAAAGGGTCAGTGTGTGTAGAATGATGTCAGCTAT-3′, | (SEQ ID NO: 14) |
| 5′-GCGGTCCCAAAAGGGTCAGTCAGATTGAGCATACTAAAAGTG-3′, | (SEQ ID NO: 15) |
| 5′-GCGGTCCCAAAAGGGTCAGTTACATGAATGACATTTACAGCA-3′, | (SEQ ID NO: 16) |
| 5′-GCGGTCCCAAAAGGGTCAGTAAGAACTGGATCAGGGAAGA-3′, | (SEQ ID NO: 17) |
| 5′-GCGGTCCCAAAAGGGTCAGTTCCTTTTGCTCACCTGTGGT-3′, | (SEQ ID NO: 18) |
| 5′-GCGGTCCCAAAAGGGTCAGTGGTCCCACTTTTTATTCTTTTGC-3′, | (SEQ ID NO: 19) |
| 5′-GCGGTCCCAAAAGGGTCAGTTGGTTTCTTAGTGTFFTGGAGTTG-3′, | (SEQ ID NO: 20) |
| 5′-GCGGTCCCAAAAGGGTCAGTTGGATCATGGGCCATGTGC-3′, | (SEQ ID NO: 21) |
| 5′-GCGGTCCCAAAAGGGTCAGTACTACCTTGCCTGCTCCAGTGG-3′, | (SEQ ID NO: 22) |
| 5′-GCGGTCCCAAAAGGGTCAGTAGGTAGCAGCTATFFTTTATGG-3′, | (SEQ ID NO: 23) |
| 5′-GCGGTCCCAAAAGGGTCAGTTAAGGGAGTCTTTTGCACAA-3′, | (SEQ ID NO: 24) |
| 5′-GCGGTCCCAAAAGGGTCAGTGCAATTTTGGATGACCTTC-3′, | (SEQ ID NO: 25) |
| 5′-GCGGTCCCAAAAGGGTCAGTTAGACAGGACTTCAACCCTC-3′, | (SEQ ID NO: 26) |
| 5′-GCGGTCCCAAAAGGGTCAGTGGTGATTATGGGAGAACTGG-3′, | (SEQ ID NO: 27) |
| 5′-GCGGTCCCAAAAGGGTCAGTATGCTTTGATGACGCTTC-3′, | (SEQ ID NO: 28) |
| 5′-GCGGTCCCAAAAGGGTCAGTTTCATTGAAAAGCCCGAC-3′, | (SEQ ID NO: 29) |
| 5′-GCGGTCCCAAAAGGGTCAGTCACCTTCTGTGTATTTTGCTG-3′, | (SEQ ID NO: 30) |
| 5′-GCGGTCCCAAAAGGGTCAGTAAGTATTGGACAACTTGTTAGTCTC-3′, | (SEQ ID NO: 31) |
| 5′-GCGGTCCCAAAAGGGTCAGTCGCCTTTCCAGTTGTATAATTT-3′. | (SEQ ID NO: 32) |

The above pairs of primers have been designed for multiplex use. Thus, one may simultaneously in a single sample amplify one or more CFTR gene segments. In preferred embodiment, five pairs of primers are used to amplify at least five CFTR gene segments. In a more preferred embodiment, ten pairs may be used and in most preferred embodiment, all 15 pairs of primers may be used.

The identify of mutations characteristics of each amplified segment for each primer pair are shown in the following table.

The table below identifies preferred primer pairs and characteristics of the amplified product.

TABLE 1
CFTR Primer Pairs and Amplicon Characteristics

| Forward Primer | Reverse Primer | Exon/Intron | Size |
|---|---|---|---|
| g14be1F | g14be24 | 14b/i14b | 149 |
| (SEQ ID NO. 11) | (SEQ ID NO. 12) | | |
| q5e12F | q5e13R | 5/i5 | 165 |
| (SEQ ID NO. 31) | (SEQ ID NO. 32) | | |
| g20e1F | g20e2R | 20 | 194 |
| (SEQ ID NO. 17) | (SEQ ID NO. 18) | | |
| q16i1F | q16i2R | 16/i16 | 200 |
| (SEQ ID NO. 25) | (SEQ ID NO. 26) | | |
| q10e10F | q10e11R | 10 | 204 |
| (SEQ ID NO. 27) | (SEQ ID NO. 28) | | |
| q21e1F | q21e2R | 21 | 215 |
| (SEQ ID NO. 13) | (SEQ ID NO. 14) | | |
| g11e1F | g11e2R | i10/11/i11 | 240 |
| (SEQ ID NO. 15) | (SEQ ID NO. 16) | | |
| g7e1F | g7e2R | 7 | 259 |
| (SEQ ID NO. 7) | (SEQ ID NO. 8) | | |
| g4e1F | g4e2R | 4/i4 | 306 |
| (SEQ ID NO. 3) | (SEQ ID NO. 4) | | |
| q3e2F | q3e2R | 3/i3 | 308 |
| (SEQ ID NO. 19) | (SEQ ID NO. 20) | | |
| q19e12F | q1913e2R | i18/19 | 310 |
| (SEQ ID NO. 29) | (SEQ ID NO. 30) | | |
| q13e2F | g13e2R | 13 | 334 |
| (SEQ ID NO. 23) | (SEQ ID NO. 24) | | |
| g9e9F | g9e9R | i8/9 | 351 |
| (SEQ ID NO. 21) | (SEQ ID NO. 22) | | |
| g19i2F | g19i3R | i19 | 389 |
| (SEQ ID NO. 5) | (SEQ ID NO. 6) | | |
| s12e1F | s12e1R | i11/12/i12 | 465 |
| (SEQ ID NO. 9) | (SEQ ID NO. 10) | | |

The nucleic acid to be amplified may be from a biological sample such as an organism, cell culture, tissue sample, and the like. The biological sample can be from a subject which includes any eukaryotic organism or animal, preferably flugi, invertebrates, insects, arachnids, fish, amphibians, reptiles, birds, marsupials and mammals. A preferred subject is a human, which may be a patient presenting to a medical provider for diagnosis or treatment of a disease. The biological sample may be obtained from a stage of life such as a fetus, young adult, adult, and the like. Particularly preferred subjects are humans being tested for the existence of a CF carrier state or disease state.

The sample to be analyzed may consist of or comprise blood, sera, urine, feces, epidermal sample, skin sample, cheek swab, sperm, amniotic fluid, cultured cells, bone marrow sample and/or chorionic villi, and the like. A biological sample may be processed to release or otherwise make available a nucleic acid for detection as described herein. Such processing may include steps of nucleic acid manipulation, e.g., preparing a cDNA by reverse transcription of RNA from the biological sample. Thus, the nucleic acid to be amplified by the methods of the invention may be DNA or RNA.

Nucleic acid may be amplified by one or more methods known in the art for copying a target nucleic acid, thereby increasing the number of copies of a selected nucleic acid sequence. Amplification may be exponential or linear. The sequences amplified in this manner form an “amplicon.” In a preferred embodiment, the amplification by the is by the polymerase chain reaction (“PCR”) (e.g., Mullis, K. et al., Cold Spring Harbor Symp. Quant. Biol. 51:263-273 (1986); Erlich H. et al., European Patent Appln. 50,424; European Patent Appln. 84,796, European Patent Application 258,017, European Patent Appln. 237,362; Mullis, K., European Patent Appln. 201,184; Mullis K. et al., U.S. Pat. No. 4,683,202; Erlich, H., U.S. Pat. No. 4,582,788; and Saiki, R. et al., U.S. Pat. No. 4,683,194). Other known nucleic acid amplification procedures that can be used include, for example, transcription-based amplification systems or isothermal amplification methods (Malek, L. T. et al., U.S. Pat. No. 5,130,238; Davey, C. et al., European Patent Application 329,822; Schuster et al., U.S. Pat. No. 5,169,766; Miller, H. I. et al., PCT appln. WO 89/06700; Kwoh, D. et al., Proc. Natl. Acad. Sci. (U.S.A.) 86:1173 (1989); Gingeras, T. R. et al., PCT application WO 88/10315; Walker, G. T. et al., Proc. Natl. Acad. Sci. (U.S.A.) 89:392-396 (1992)). Amplification may be performed to with relatively similar levels of each primer of a primer pair to generate an double stranded amplicon. However, asymmetric PCR may be used to amplify predominantly or exclusively a single stranded product as is well known in the art (e.g., Poddar et al. Molec. And Cell. Probes 14:25-32 (2000)). This can be achieved for each pair of primers by reducing the concentration of one primer significantly relative to the other primer of the pair (e.g. 100 fold difference). Amplification by asymmetric PCR is generally linear. One of ordinary skill in the art would know that there are many other useful methods that can be employed to amplify nucleic acid with the invention primers (e.g., isothermal methods, rolling circle methods, etc.), and that such methods may be used either in place of, or together with, PCR methods. Persons of ordinary skill in the art also will readily acknowledge that enzymes and reagents necessary for amplifying nucleic acid sequences through the polymerase chain reaction, and techniques and procedures for performing PCR, are well known. The examples below illustrate a standard protocol for performing PCR and the amplification of nucleic acid sequences that correlate with or are indicative of cystic fibrosis.

In another aspect, the present invention provides methods of detecting a cystic fibrosis genotype in a biological sample. The methods comprise amplifying nucleic acids in a biological sample of the subject and identifying the presence or absence of one or more mutant cystic fibrosis nucleic acid sequences in the amplified nucleic acid. Accordingly, the present invention provides a method of determining the presence or absence of one or more mutant cystic fibrosis nucleic acid sequences in a nucleic acid containing sample, comprising: contacting said sample with reagents suitable for nucleic acid amplification including one or more pairs of nucleic acid primers flanking one or more predetermined nucleic acid sequences that are correlated with cystic fibrosis, amplifying said predetermined nucleic acid sequence(s), if present, to provide an amplified sample; and identifying the presence or absence of said one or more predetermined sequences in said amplified sample, whereby the presence or absence of said one or more mutant cystic fibrosis nucleic acid sequences is determined; wherein said pairs of nucleic acid primers are selected from the group consisting of:

| | |
|---|---|
| 5′-GCGGTCCCAAAAGGGTCAGTTGTAGGAAGTCACCAAAG-3′ | (SEQ ID NO: 3) |
| and | |
| 5′-GCGGTCCCAAAAGGGTCAGTCGATACAGAATATATGTGCC-3′, | (SEQ ID NO: 4) |
| 5′-GCGGTCCCAAAAGGGTCAGTGAATCATTCAGTGGGTATAAGCAG-3′ | (SEQ ID NO: 5) |
| and | |
| 5′-GCGGTCCCAAAAGGGTCAGTCTTCAATGCACCTCCTCCC-3′, | (SEQ ID NO: 6) |
| 5′-GCGGTCCCAAAAGGGTCAGTAGATACTTCAATAGCTCAGCC-3′ | (SEQ ID NO: 7) |
| and | |
| 5′-GCGGTCCCAAAAGGGTCAGTGGTACATTACCTGTATTTTGTTT-3′, | (SEQ ID NO: 8) |
| 5′-GCGGTCCCAAAAGGGTCAGTGTGAATCGATGTGGTGACCA-3′ | (SEQ ID NO: 9) |
| and | |
| 5′-GCGGTCCCAAAAGGGTCAGTCTGGTTTAGCATGAGGCGGT-3′, | (SEQ ID NO: 10) |
| 5′-GCGGTCCCAAAAGGGTCAGTTTGGTTGTGCTGTGGCTCCT-3′ | (SEQ ID NO: 11) |
| and | |
| 5′-GCGGTCCCAAAAGGGTCAGTACAATACATACAAACATAGTGG-3′, | (SEQ ID NO: 12) |
| 5′-GCGGTCCCAAAAGGGTCAGTGAAAGTATTTATTTTTTCTGGAAC-3′ | (SEQ ID NO: 13) |
| and | |
| 5′-GCGGTCCCAAAAGGGTCAGTGTGTGTAGAATGATGTCAGCTAT-3′, | (SEQ ID NO: 14) |
| 5′-GCGGTCCCAAAAGGGTCAGTCAGATTGAGCATACTAAAAGTG-3′ | (SEQ ID NO: 15) |
| and | |
| 5′-GCGGTCCCAAAAGGGTCAGTTACATGAATGACATTTACAGCA-3′, | (SEQ ID NO: 16) |
| 5′-GCGGTCCCAAAAGGGTCAGTAAGAACTGGATCAGGGAAGA-3′ | (SEQ ID NO: 17) |
| and | |
| 5′-GCGGTCCCAAAAGGGTCAGTTCCTTTTGCTCACCTGTGGT-3′, | (SEQ ID NO: 18) |
| 5′-GCGGTCCCAAAAGGGTCAGTGGTCCCACTTTTTATTCTTTTGC-3′ | (SEQ ID NO: 19) |
| and | |
| 5′-GCGGTCCCAAAAGGGTCAGTTGGTTTCTTAGTGTTTGGAGTTG-3′, | (SEQ ID NO: 20) |
| 5′-GCGGTCCCAAAAGGGTCAGTTGGATCATGGGCCATGTGC-3′ | (SEQ ID NO: 21) |
| and | |
| 5′-GCGGTCCCAAAAGGGTCAGTACTACCTTGCCTGCTCCAGTGG-3′, | (SEQ ID NO: 22) |
| 5′-GCGGTCCCAAAAGGGTCAGTAGGTAGCAGCTATTTTTATGG-3′ | (SEQ ID NO: 23) |
| and | |
| 5′-GCGGTCCCAAAAGGGTCAGTTAAGGGAGTCTTTTGCACAA-3′, | (SEQ ID NO: 24) |
| 5′-GCGGTCCCAAAAGGGTCAGTGCAATTTTGGATGACCTTC-3′ | (SEQ ID NO: 25) |
| and | |
| 5′-GCGGTCCCAAAAGGGTCAGTTAGACAGGACTTCAACCCTC-3′, | (SEQ ID NO: 26) |
| 5′-GCGGTCCCAAAAGGGTCAGTGGTGATTATGGGAGAACTGG-3′ | (SEQ ID NO: 27) |
| and | |
| 5′-GCGGTCCCAAAAGGGTCAGTATGCTTTGATGACGCTTC-3′, | (SEQ ID NO: 28) |
| 5′-GCGGTCCCAAAAGGGTCAGTTTCAGTTTGAAAAGCCCGAC-3′ | (SEQ ID NO: 29) |
| and | |
| 5′-GCGGTCCCAAAAGGGTCAGTCACCTTCTGTGTATTTTGCTG-3′, | (SEQ ID NO: 30) |
| and | |
| 5′-GCGGTCCCAAAAGGGTCAGTAAGTATTGGACAACTTGTTAGTCTC-3′ | (SEQ ID NO: 31) |
| and | |
| 5′-GCGGTCCCAAAAGGGTCAGTCGCCTTTCCAGTTGTATAATTT-3′. | (SEQ ID NO: 32) |

One may analyze the amplified product for the presence of absence of any of a number of mutant CF sequences that may be present in the sample nucleic acid. As already discussed, numerous mutations in the CFTR gene have been associated with CF carrier and disease states. For example, a three base pair deletion leading to the omission of a phenylalanine residue in the gene product has been determined to correspond to the mutations of the CF gene in approximately 70% of the patients affected by CF. The table below identifies preferred CF sequences and identifies which of the primer pairs of the invention may be used to amplify the sequence.

TABLE 2
CFTR mutations that may be detected in amplified product
using as the primer pair SEQ ID NO: 19 and 20.

| Name | Nucleotide change | Exon | Consequence |
|---|---|---|---|
| 297 − 3C −> T | C to T at 297 − 3 | intron 2 | mRNA splicing |
| | | | defect? |
| E56K | G to A at 298 | 3 | Glu to Lys at 56 |
| 300delA | deletion of A at 300 | 3 | Frameshift |
| W57R | T to C at 301 | 3 | Trp to Arg at 57 |
| W57G | T to G at 301 | 3 | Trp to Gly at 57 |
| W57X(TAG) | G to A at 302 | 3 | Trp to Stop at 57 |
| W57X(TGA) | G to A at 303 | 3 | Trp to Stop at 57 |
| D58N | G to A at 304 | 3 | Asp to Asn at 58 |
| D58G | A to G at 305 | 3 | Asp to Gly at 58 |
| 306insA | insertion of A at 306 | 3 | Frameshift |
| 306delTAGA | deletion of TAGA from | 3 | Frameshift |
| | 306 | | |
| E60L | G to A at 310 | 3 | Glu to Leu at 60 |
| E60X | G to T at 310 | 3 | Glu to Stop at 60 |
| E60K | G to A at 310 | 3 | Glu to Lys at 60 |
| N66S | A to G at 328 | 3 | Asn to Ser at 66 |
| P67L | C to T at 332 | 3 | Pro to Leu at 67 |
| K68E | A to G at 334 | 3 | Lys to Glu at 68 |
| K68N | A to T at 336 | 3 | Lys to Asn at 68 |
| A72T | G to A at 346 | 3 | Ala to Thr at 72 |
| A72D | C to A at 347 | 3 | Ala to Asp at 72 |
| 347delC | deletion of C at 347 | 3 | Frameshift |
| R74W | C to T at 352 | 3 | Arg to Trp at 74 |
| R74Q | G to A at 353 | 3 | Arg to Gln at 74 |
| R75X | C to T at 355 | 3 | Arg to Stop at 75 |
| R75L | G to T at 356 | 3 | Arg to Leu at 75 |
| 359insT | insertion of T after 359 | 3 | Frameshift |
| 360delT | deletion of T at 360 | 3 | Frameshift |
| W79R | T to C at 367 | 3 | Trp to Arg at 79 |
| W79X | G to A at 368 | 3 | Trp to Stop at 79 |
| G85E | G to A at 386 | 3 | Gly to Glu at 85 |
| G85V | G to T at 386 | 3 | Gly to Val at 85 |
| F87L | T to C at 391 | 3 | Phe to Leu at 87 |
| 394delTT | deletion of TT from 394 | 3 | frameshift |
| L88S | T to C at 395 | 3 | Leu to Ser at 88 |
| L88X(T −> A) | T to A at 395 | 3 | Leu to Stop at 88 |
| L88X(T −> G) | T to G at 395 | 3 | Leu to Stop at 88 |
| Y89C | A to G at 398 | 3 | Tyr to Cys at 89 |
| L90S | T to C at 401 | 3 | Leu to Ser at 90 |
| G91R | G to A at 403 | 3 | Gly to Arg at 91 |
| 405 + 1G −> A | G to A at 405 + 1 | intron 3 | mRNA splicing |
| | | | defect |
| 405 + 3A −> C | A to C at 405 + 3 | intron 3 | mRNA splicing |
| | | | defect? |
| 405 + 4A −> G | A to G at 405 + 4 | intron 3 | mRNA splicing |
| | | | defect? |

TABLE 3
CFTR mutations that may be detected in amplified product
using as the primer pair SEQ ID NO: 3 and 4.

| Name | Nucleotide change | Exon | Consequence |
|---|---|---|---|
| A96E | C to A at 419 | 4 | Ala to Glu at 96 |
| Q98X | C to T at 424 | 4 | Gln to Stop at 98 |
| | | | (Pakistani |
| | | | specific?) |
| Q98P | A to C at 425 | 4 | Gln to Pro at 98 |
| Q98R | A to G at 425 | 4 | Gln to Arg at 98 |
| P99L | C to T at 428 | 4 | Pro to Leu at 99 |
| L101X | T to G at 434 | 4 | Leu to Stop at 101 |
| 435insA | insertion of A | 4 | Frameshift |
| | after 435 | | |
| G103X | G to T at 439 | 4 | Gly to Stop at 103 |
| 441delA | deletion of A | 4 | Frameshift |
| | at 441 and T | | |
| | to A at 486 | | |
| 444delA | deletion of A | 4 | Frameshift |
| | at 444 | | |
| I105N | T to A at 446 | 4 | Ile to Asn at 105 |
| 451del8 | deletion of | 4 | Frameshift |
| | GCTTCCTA from | | |
| | 451 | | |
| S108F | C to T at 455 | 4 | Ser to Phe at 108 |
| 457TAT −> G | TAT to G at 457 | 4 | Frameshift |
| Y109N | T to A at 457 | 4 | Tyr to Asn at 109 |
| 458delAT | deletion of AT | 4 | Frameshift |
| | at 458 | | |
| Y109C | A to G at 458 | 4 | Tyr to Cys at 109 |
| 460delG | deletion of G | 4 | Frameshift |
| | at 460 | | |
| D110Y | G to T at 460 | 4 | Asp to Tyr at 110 |
| D110H | G to C at 460 | 4 | Asp to His at 110 |
| D110E | C to A at 462 | 4 | Asp to Glu at 110 |
| P111A | C to G at 463 | 4 | Pro to Ala at 111 |
| P111L | C to T at 464 | 4 | Pro to Leu at 111 |
| [delta]E115 | 3 bp deletion of | 4 | deletion of Glu |
| | 475-477 | | at 115 |
| E116Q | G to C at 478 | 4 | Glu to Gln at 116 |
| E116K | G to A at 478 | 4 | Glu to Lys at 116 |
| R117C | C to T at 481 | 4 | Arg to Cys at 117 |
| R117P | G to C at 482 | 4 | Arg to Pro at 117 |
| R117L | G to T at 482 | 4 | Arg to Leu at 117 |
| R117H | G to A at 482 | 4 | Arg to His at 117 |
| I119V | A to G at 487 | 4 | Iso to Val at 119 |
| A120T | G to A at 490 | 4 | Ala to Thr at 120 |
| Y122X | T to A at 498 | 4 | Tyr to Stop at 122 |
| I125T | T to C at 506 | 4 | Ile to Thr at 125 |
| G126D | G to A at 509 | 4 | Gly to Asp at 126 |
| L127X | T to G at 512 | 4 | Leu to Stop at 127 |
| 525delT | deletion of T | 4 | Frameshift |
| | at 525 | | |
| 541del4 | deletion of | 4 | Frameshift |
| | CTCC from 541 | | |
| 541delC | deletion of C | 4 | Frameshift |
| | at 541 | | |
| L137R | T to G at 542 | 4 | Leu to Arg at 137 |
| L137H | T to A at 542 | 4 | Leu to His at 137 |
| L138ins | insertion of CTA, | 4 | insertion of |
| | TAC or ACT at | | leucine at 138 |
| | nucleotide 544, | | |
| | 545 or 546 | | |
| 546insCTA | insertion of CTA | 4 | Frameshift |
| | at 546 | | |
| 547insTA | insertion of TA | 4 | Frameshift |
| | after 547 | | |
| H139L | A to T at 548 | 4 | His to Leu at 548 |
| H139R | A to G at 548 | 4 | His to Arg at 139 |
| P140S | C to T at 550 | 4 | Pro to Ser at 140 |
| P140L | C to T at 551 | 4 | Pro to Leu at 140 |
| 552insA | insertion of A | 4 | Frameshift |
| | after 552 | | |
| A141D | C to A at 554 | 4 | Ala to Asp at 141 |
| 556delA | deletion of A | 4 | Frameshift |
| | at 556 | | |
| 557delT | deletion of T | 4 | Frameshift |
| | at 557 | | |
| 565delC | deletion of C | 4 | Frameshift |
| | at 565 | | |
| H146R | A to G at 569 | 4 | His to Arg at 146 |
| | | | (CBAVD) |
| 574delA | deletion of A | 4 | Frameshift |
| | at 574 | | |
| I148N | T to A at 575 | 4 | Ile to Asn at 148 |
| I148T | T to C at 575 | 4 | Ile to Thr at 148 |
| G149R | G to A at 577 | 4 | Gly to Arg at 149 |
| Q151X | C to T at 583 | 4 | Gln to Stop at 151 |
| M152V | A to G at 586 | 4 | Met to Val at 152 |
| | | | (mutation?) |
| M152R | T to G at 587 | 4 | Met to Arg at 152 |
| 591del18 | deletion of 18 | 4 | deletion of 6 amino |
| | bp from 591 | | acids from the CFTR |
| | | | protein |
| A155P | G to C at 595 | 4 | Ala to Pro at 155 |
| S158R | A to C at 604 | 4 | Ser to Arg at 158 |
| 605insT | insertion of T | 4 | Frameshift |
| | after 605 | | |
| L159X | T to A at 608 | 4 | Leu to Stop at 159 |
| Y161D | T to G at 613 | 4 | Tyr to Asp at 161 |
| Y161N | T to A at 613 | 4 | Tyr to Asn at 161 |
| Y161S | A to C at 614 | 4 | Tyr to Ser at 161 |
| | (together with | | |
| | 612T/A) | | |
| K162E | A to G at 616 | 4 | Lys to Glu at 162 |
| 621G −> A | G to A at 621 | 4 | mRNA splicing defect |
| 621 + 1G −> T | G to T at 621 + 1 | intron 4 | mRNA splicing defect |
| 621 + 2T −> C | T to C at 621 + 2 | intron 4 | mRNA splicing defect |
| 621 + 2T −> G | T to G at 621 + 2 | intron 4 | mRNA splicing defect |
| 621 + 3A −> G | A to G at 621 + 3 | intron 4 | mRNA splicing defect |

TABLE 4
CFTR mutations that may be detected in amplified product
using as the primer pair SEQ ID NO: 31 and 32.

| Name | Nucleotide_change | Exon | Consequence |
|---|---|---|---|
| 681delC | deletion of C at 681 | 5 | Frameshift |
| N186K | C to A at 690 | 5 | Asn to Lys at 186 |
| N187K | C to A at 693 | 5 | Asn to Lys at 187 |
| [delta]D192 | deletion of TGA or | 5 | deletion of Asp |
| | GAT from 706 or 707 | | at 192 |
| D192N | G to A at 706 | 5 | Asp to Asn at 192 |
| D192G | A to G at 707 | 5 | Asp to Gly at 192 |
| E193K | G to A at 709 | 5 | Glu to Lys at 193 |
| E193X | G to T at 709 | 5 | Glu to Stop at 193 |
| 711 + 1G −> T | G to T at 711 + 1 | intron 5 | mRNA splicing |
| | | | defect |
| 711 + 3A −> G | A to G at 711 + 3 | intron 5 | mRNA splicing |
| | | | defect |
| 711 + 3A −> C | A to C at 711 + 3 | intron 5 | mRNA splicing |
| | | | defect |
| 711 + 3A −> T | A to T at 711 + 3 | intron 5 | mRNA splicing |
| | | | defect? |
| 711 + 5G −> A | G to A at 711 + 5 | intron 5 | mRNA splicing |
| | | | defect |
| 711 + 34A −> G | A to G at 711 + 34 | intron 5 | mRNA splicing |
| | | | defect? |

TABLE 5
CFTR mutations that may be detected in amplified product
using as the primer pair SEQ ID NO: 7 and 8.

| Name | Nucleotide_change | Exon | Consequence |
|---|---|---|---|
| [delta]F311 | deletion of 3 bp between | 7 | deletion of Phe310, |
| | 1059 and 1069 | | 311 or 312 |
| F311L | C to G at 1065 | 7 | Phe to Leu at 311 |
| G314R | G to C at 1072 | 7 | Gly to Arg at 314 |
| G314E | G to A at 1073 | 7 | Gly to Glu at 314 |
| G314V | G to T at 1073 | 7 | Gly to Val at 324 |
| F316L | T to G at 1077 | 7 | Phe to Leu at 316 |
| 1078delT | deletion of T at 1078 | 7 | Frameshift |
| V317A | T to C at 1082 | 7 | Val to Ala at 317 |
| L320V | T to G at 1090 | 7 | Leu to Val at 320 |
| | | | CAVD |
| L320X | T to A at 1091 | 7 | Leu to Stop at 320 |
| L320F | A to T at 1092 | 7 | Leu to Phe at 320 |
| V322A | T to C at 1097 | 7 | Val to Ala at 322 |
| | | | (mutation?) |
| 1112delT | deletion of T at 1112 | 7 | Frameshift |
| L327R | T to G at 1112 | 7 | Leu to Arg at 327 |
| 1119delA | deletion of A at 1119 | 7 | Frameshift |
| G330X | G to T at 1120 | 7 | Gly to Stop at 330 |
| R334W | C to T at 1132 | 7 | Arg to Trp at 334 |
| R334Q | G to A at 1133 | 7 | Arg to Gln at 334 |
| R334L | G to T at 1133 | 7 | Arg to Leu at 334 |
| 1138insG | insertion of G after 1138 | 7 | Frameshift |
| I336K | T to A at 1139 | 7 | Ile to Lys at 336 |
| T338I | C to T at 1145 | 7 | Thr to Ile at 338 |
| 1150delA | deletion of A at 1150 | 7 | Frameshift |
| 1154insTC | insertion of TC after | 7 | Frameshift |
| | 1154 | | |
| 1161insG | insertion of G after 1161 | 7 | Frameshift |
| 1161delC | deletion of C at 1161 | 7 | Frameshift |
| L346P | T to C at 1169 | 7 | Leu to Pro at 346 |
| R347C | C to T at 1171 | 7 | Arg to Cys at 347 |
| R347H | G to A at 1172 | 7 | Arg to His at 347 |
| R347L | G to T at 1172 | 7 | Arg to Leu at 347 |
| R347P | G to C at 1172 | 7 | Arg to Pro at 347 |
| M348K | T to A at 1175 | 7 | Met to Lys at 348 |
| A349V | C to T at 1178 | 7 | Ala to Val at 349 |
| R352W | C to T at 1186 | 7 | Arg to Trp at 352 |
| R352Q | G to A at 1187 | 7 | Arg to Gln at 352 |
| Q353X | C to T at 1189 | 7 | Gln to Stp at 353 |
| Q353H | A to C at 1191 | 7 | Gln to His at 353 |
| 1199delG | deletion of G at 1199 | 7 | Frameshift |
| W356X | G to A at 1200 | 7 | Trp to Stop at 356 |
| Q359K/T360K | C to A at 1207 and C to | 7 | Glu to Lys at 359 |
| | A at 1211 | | and Thr to Lys at |
| | | | 360 |
| Q359R | A to G at 1208 | 7 | Gln to Arg at 359 |
| 1213delT | deletion of T at 1213 | 7 | Frameshift |
| W361R(T −> C) | T to C at 1213 | 7 | Trp to Arg at 361 |
| W361R(T −> A) | T to A at 1213 | 7 | Trp to Arg at 361 |
| 1215delG | deletion of G at 1215 | 7 | Frameshift |
| 1221delCT | deletion of CT from | 7 | Frameshift |
| | 1221 | | |
| S364P | T to C at 1222 | 7 | Ser to Pro at 364 |
| L365P | T to C at 1226 | 7 | Leu to Pro at 365 |

TABLE 6
CFTR mutations that may be detected in amplified product
using as the primer pair SEQ ID NO: 21 and 22.

| Name | Nucleotide_change | Exon | Consequence |
|---|---|---|---|
| 1342 − | TTT to G at | intron 8 | mRNA splicing |
| 11TTT −> G | 1342 − 11 | | defect? |
| 1342 − 2delAG | deletion of AG | intron 8 | Frameshift |
| | from 1342 − 2 | | |
| 1342 − | A to C at 1342 − 2 | intron 8 | mRNA splicing |
| 2A −> C | | | defect |
| 1342 − | G to C at 1342 − 1 | intron 8 | mRNA splicing |
| 1G −> C | | | defect |
| E407V | A to T at 1352 | 9 | Glu to Val at 407 |
| 1366delG | deletion of G | 9 | Frameshift |
| | at 1366 | | |
| 1367delC | deletion of C | 9 | Frameshift |
| | at 1367 | | |
| 1367del5 | deletion of | 9 | Frameshift |
| | CAAAA at 1367 | | |
| Q414X | C to T at 1372 | 9 | Gln to Stop at 414 |
| N418S | A to G at 1385 | 9 | Asn to Ser at 418 |
| G424S | G to A at 1402 | 9 | Gly to Ser at 424 |
| S434X | C to G at 1433 | 9 | Ser to Stop at 434 |
| D443Y | G to T at 1459 | 9 | Asp to Tyr at 443 |
| 1460delAT | deletion of AT | 9 | Frameshift |
| | from 1460 | | |
| 1461ins4 | insertion of AGAT | 9 | Frameshift |
| | after 1461 | | |
| I444S | T to G at 1463 | 9 | Ile to Ser at 444 |
| 1471delA | deletion of A | 9 | Frameshift |
| | at 1471 | | |
| Q452P | A to C at 1487 | 9 | Gln to Pro at 452 |
| [delta]L453 | deletion of 3 | 9 | deletion of Leu |
| | bp between | | at 452 or 454 |
| | 1488 and 1494 | | |
| A455E | C to A at 1496 | 9 | Ala to Glu at 455 |
| V456F | G to T at 1498 | 9 | Val to Phe at 456 |

TABLE 7
CFTR mutations that may be detected in amplified product
using as the primer pair SEQ ID NO: 27 and 28.

| Name | Nucleotide_change | Exon | Consequence |
|---|---|---|---|
| G480C | G to T at 1570 | 10 | Gly to Cys at 480 |
| G480D | G to A at 1570 | 10 | Gly to Asp at 480 |
| G480S | G to A at 1570 | 10 | Gly to Ser at 480 |
| 1571delG | deletion of G at 1571 | 10 | Frameshift |
| 1576insT | insertion of T at 1576 | 10 | Framshift |
| H484Y | C to T at 1582 | 10 | His to Tyr at 484 |
| | | | (CBAVD?) |
| H484R | A to G at 1583 | 10 | His to Arg at 484 |
| S485C | A to T at 1585 | 10 | Ser to Cys at 485 |
| G486X | G to T at 1588 | 10 | Glu to Stop at 486 |
| S489X | C to A at 1598 | 10 | Ser to Stop at 489 |
| 1601delTC | deletion of TC from | 10 | Frameshift |
| | 1601 or CT from 1602 | | |
| C491R | T to C at 1603 | 10 | Cys to Arg at 491 |
| S492F | C to T at 1607 | 10 | Ser to Phe at 492 |
| Q493X | C to T at 1609 | 10 | Gln to Stop at 493 |
| 1609delCA | deletion of CA from | 10 | Frameshift |
| | 1609 | | |
| Q493R | A to G at 1610 | 10 | Gln to Arg at 493 |
| 1612delTT | deletion of TT from | 10 | Frameshift |
| | 1612 | | |
| W496X | G to A at 1619 | 10 | Trp to Stop at 496 |
| P499A | C to G at 1627 | 10 | Pro to Ala at 499 |
| | | | (CBAVD) |
| T501A | A to G at 1633 | 10 | Thr to Ala at 501 |
| I502T | T to C at 1637 | 10 | Ile to Thr at 502 |
| I502N | T to A at 1637 | 10 | Ile to Asn at 502 |
| E504X | G to T at 1642 | 10 | Glu to Stop at 504 |
| E504Q | G to C at 1642 | 10 | Glu to Gln at 504 |
| I506L | A to C at 1648 | 10 | Ile to Leu at 506 |
| [delta]I507 | deletion of 3 bp between | 10 | deletion of Ile506 |
| | 1648 and 1653 | | or Ile507 |
| I506S | T to G at 1649 | 10 | Ile to Ser at 506 |
| I506T | T to C at 1649 | 10 | Ile to Thr at 506 |
| [delta]F508 | deletion of 3 bp between | 10 | deletion of Phe |
| | 1652 and 1655 | | at 508 |
| F508S | T to C at 1655 | 10 | Phe to Ser at 508 |
| D513G | A to G at 1670 | 10 | Asp to Gly at 513 |
| | | | (CBAVD) |
| 1677delTA | deletion of TA from | 10 | frameshift |
| | 1677 | | |
| Y517C | A to G at 1682 | 10 | Tyr to Cys at 517 |

TABLE 8
CFTR mutations that may be detected in amplified product
using as the primer pair SEQ ID NO: 15 and 16.

| Name | Nucleotide_change | Exon | Consequence |
|---|---|---|---|
| 1716 − 1G −> A | G to A at 1716 − 1 | intron 10 | mRNA splicing defect |
| 1717 − 8G −> A | G to A at 1717 − 8 | intron 10 | mRNA splicing defect? |
| 1717 − 3T −> G | T to G at 1717 − 3 | intron 10 | mRNA splicing defect? |
| 1717 − 2A −> G | A to G at 1717 − 2 | intron 10 | mRNA splicing defect |
| 1717 − 1G −> A | G to A at 1717 − 1 | intron 10 | mRNA splicing defect |
| D529H | G to C at 1717 | 11 | Asp to His at 529 |
| 1717 − 9T −> A | T to A at 1717 − 9 | intron 10 | mRNA splicing mutation? |
| A534E | C to A at 1733 | 11 | Ala to Glu at 534 |
| 1742delAC | deletion of AC from | 11 | Frameshift |
| | 1742 | | |
| I539T | T to C at 1748 | 11 | Ile to Thr at 539 |
| 1749insTA | insertion of TA at 1749 | 11 | frameshift resulting in |
| | | | premature termination at 540 |
| G542X | G to T at 1756 | 11 | Gly to Stop at 542 |
| G544S | G to A at 1762 | 11 | Gly to Ser at 544 |
| G544V | G to T at 1763 | 11 | Gly to Val at 544 (CBAVD) |
| 1774delCT | deletion of CT from | 11 | Frameshift |
| | 1774 | | |
| S549R(A −> C) | A to C at 1777 | 11 | Ser to Arg at 549 |
| S549I | G to T at 1778 | 11 | Ser to Ile at 549 |
| S549N | G to A at 1778 | 11 | Ser to Asn at 549 |
| S549R(T −> G) | T to G at 1779 | 11 | Ser to Arg at 549 |
| G550X | G to T at 1780 | 11 | Gly to Stop at 550 |
| G550R | G to A at 1780 | 11 | Gly to Arg at 550 |
| 1782delA | deletion of A at 1782 | 11 | Frameshift |
| G551S | G to A at 1783 | 11 | Gly to Ser at 551 |
| 1784delG | deletion of G at 1784 | 11 | Frameshift |
| G551D | G to A at 1784 | 11 | Gly to Asp at 551 |
| Q552X | C to T at 1786 | 11 | Gln to Stop at 552 |
| Q552K | C to A at 1786 | 11 | Gln to Lys |
| 1787delA | deletion of A at position | 11 | frameshift, stop codon at 558 |
| | 1787 or 1788 | | |
| R553G | C to G at 1789 | 11 | Arg to Gly at 553 |
| R553X | C to T at 1789 | 11 | Arg to Stop at 553 |
| R553Q | G to A at 1790 | 11 | Arg to Gln at 553 (associated |
| | | | with [delta]F508; |
| R555G | A to G at 1795 | 11 | Arg to Gly at 555 |
| I556V | A to G at 1798 | 11 | Ile to Val at 556 (mutation?) |
| 1802delC | deletion of C at 1802 | 11 | Frameshift |
| L558S | T to C at 1805 | 11 | Leu to Ser at 558 |
| 1806delA | deletion of A at 1806 | 11 | Frameshift |
| A559T | G to A at 1807 | 11 | Ala to Thr at 559 |
| A559E | C to A at 1808 | 11 | Ala to Glu at 559 |
| R560T | G to C at 1811 | 11 | Arg to Thr at 560; mRNA |
| | | | splicing defect? |
| R560K | G to A at 1811 | 11 | Arg to Lys at 560 |
| 1811 + 1G −> C | G to C at 1811 + 1 | intron 11 | mRNA splicing defect |
| 1811 + 1.6kbA −> G | A to G at 1811 + 1.2kb | intron 11 | creation of splice donor site |
| 1811 + 18G −> A | G to A at 1811 + 18 | intron 11 | mRNA splicing defect? |

TABLE 9
CFTR mutations that may be detected in amplified product
using as the primer pair SEQ ID NO: 9 and 10.

| Name | Nucleotide change | Exon | Consequence |
|---|---|---|---|
| 1812 − 1G −> A | G to A at 1812 −1 | intron 11 | mRNA splicing defect |
| R560S | A to C at 1812 | 12 | Arg to Ser at 560 |
| 1813insC | insertion of C after 1813 | 12 | Frameshift |
| | (or 1814) | | |
| A561E | C to A at 1814 | 12 | Ala to Glu at 561 |
| V562I | G to A at 1816 | 12 | Val to Ile at 562 |
| V562L | G to C at 1816 | 12 | Val to Leu at 562 |
| Y563D | T to G at 1819 | 12 | Tyr to Asp at 563 |
| Y563N | T to A at 1819 | 12 | Tyr to Asn at 563 |
| Y563C | A to G at 1821 | 12 | Tyr to Cys at 563 |
| 1833delT | deletion of T at 1833 | 12 | Frameshift |
| L568X | T to A at 1835 | 12 | Leu to Stop at 568 |
| L568F | G to T at 1836 | 12 | Leu to Phe at 568 |
| | | | (CBAVD?) |
| Y569D | T to G at 1837 | 12 | Tyr to Asp at 569 |
| Y569H | T to C at 1837 | 12 | Tyr to His at 569 |
| Y569C | A to G at 1838 | 12 | Tyr to Cys at 569 |
| V569X | T to A at 1839 | 12 | Tyr to Stop at 569 |
| L571S | T to C at 1844 | 12 | Leu to Ser at 571 |
| 1845delAG/1846d | deletion of AG at 1845 | 12 | Frameshift |
| elGA | or GA at 1846 | | |
| D572N | G to A at 1846 | 12 | Asp to Asn at 572 |
| P574H | C to A at 1853 | 12 | Pro to His at 574 |
| G576X | G to T at 1858 | 12 | Gly to Stop at 576 |
| G576A | G to C at 1859 | 12 | Gly to Ala at 576 (CAVD) |
| Y577F | A to T at 1862 | 12 | Tyr to Phe at 577 |
| D579Y | G to T at 1867 | 12 | Asp to Tyr at 579 |
| D579G | A to G at 1868 | 12 | Asp to Gly at 579 |
| D579A | A to C at 1868 | 12 | Asp to Ala at 579 |
| 1870delG | deletion of G at 1870 | 12 | Frameshift |
| 1874insT | insertion of T between | 12 | Frameshift |
| | 1871 and 1874 | | |
| T582R | C to G at 1877 | 12 | Thr to Arg at 582 |
| T582I | C to T at 1877 | 12 | Thr to Ile at 582 |
| E585X | G to T at 1885 | 12 | Glu to Stop at 585 |
| S589N | G to A at 1898 | 12 | Ser to Asn at 589 (mRNA |
| | | | splicing defect?) |
| S589I | G to T at 1898 | 12 | Ser to Ile at 589 (splicing?) |
| 1898 + 1G −> A | G to A at 1898 + 1 | intron 12 | mRNA splicing defect |
| 1898 + 1G −> C | G to C at 1898 + 1 | intron 12 | mRNA splicing defect |
| 1898 + 1G −> T | G to T at 1898 + 1 | intron 12 | mRNA splicing defect |
| 1898 + 3A −> G | A to G at 1898 + 3 | intron 12 | mRNA splicing defect? |
| 1898 + 3A −> C | A to C at 1898 + 3 | intron 12 | mRNA splicing defect? |
| 1898 + 5G −> A | G to A at 1898 + 5 | intron 12 | mRNA splicing defect |
| 1898 + 5G −> T | G to T at 1898 + 5 | intron 12 | mRNA splicing defect |
| 1898 + 73T −> G | T to G at 1898 + 73 | intron 12 | mRNA splicing defect? |

TABLE 10
CFTR mutations that may be detected in amplified product
using as the primer pair SEQ ID NO: 23 and 24.

| Name | Nucleotide_change | Exon | Consequence |
|---|---|---|---|
| 1918delGC | deletion of GC from | 13 | Frameshift |
| | 1918 | | |
| 1924del7 | deletion of 7 bp | 13 | Frameshift |
| | (AAACTA) from 1924 | | |
| R600G | A to G at 1930 | 13 | Arg to Gly at 600 |
| I601F | A to T at 1933 | 13 | Ile to Phe at 601 |
| V603F | G to T at 1939 | 13 | Val to Phe at 603 |
| T604I | C to T at 1943 | 13 | Thr to Ile at 604 |
| 1949del84 | deletion of 84 bp from | 13 | deletion of 28 a.a. |
| | 1949 | | (Met607 to Gln634) |
| H609R | A to G at 1958 | 13 | His to Arg at 609 |
| L610S | T to C at 1961 | 13 | Leu to Ser at 610 |
| A613T | G to A at 1969 | 13 | Ala to Thr at 613 |
| D614Y | G to T at 1972 | 13 | Asp to Tyr 614 |
| D614G | A to G at 1973 | 13 | Asp to Gly at 614 |
| I618T | T to C at 1985 | 13 | Ile to Thr at 618 |
| L619S | T to C at 1988 | 13 | Leu to Ser at 619 |
| H620P | A to C at 1991 | 13 | His to Pro at 620 |
| H620Q | T to G at 1992 | 13 | His to Gln at 620 |
| G622D | G to A at 1997 | 13 | Gly to Asp at 622 |
| | | | (oligospermia) |
| G628R(G −> A) | G to A at 2014 | 13 | Gly to Arg at 628 |
| G628R(G −> C) | G to C at 2014 | 13 | Gly to Arg at 628 |
| L633P | T to C at 2030 | 13 | Leu to Pro at 633 |
| Q634X | T to A at 2032 | 13 | Gln to Stop at 634 |
| L636P | T to C at 2039 | 13 | Leu to Pro at 636 |
| Q637X | C to T at 2041 | 13 | Gln to Stop at 637 |
| 2043delG | deletion of G at 2043 | 13 | Frameshift |
| 2051delTT | deletion of TT from | 13 | Frameshift |
| | 2051 | | |
| 2055del9 −> A | deletion of 9 bp | 13 | Frameshift |
| | CTCAAAACT to A at | | |
| | 2055 | | |
| D648V | A to T at 2075 | 13 | Asp to Val at 648 |
| D651N | G to A at 2083 | 13 | Asp to Asn at 651 |
| E656X | T to G at 2098 | 13 | Glu to Stop at 656 |
| 2108delA | deletion of A at 2108 | 13 | Frameshift |
| 2109del9 −> A | deletion of 9 bp from | 13 | Frameshift |
| | 2109 and insertion of A | | |
| 2113delA | deletion of A at 2113 | 13 | Frameshift |
| 2116delCTAA | deletion of CTAA at | 13 | Frameshift |
| | 2116 | | |
| 2118del4 | deletion of AACT from | 13 | Frameshift |
| | 2118 | | |
| E664X | G to T at 2122 | 13 | Glu to Stop at 664 |
| T665S | A to T at 2125 | 13 | Thr to Ser at 665 |
| 2141insA | insertion of A after 2141 | 13 | Frameshift |
| 2143delT | deletion of T at 2143 | 13 | Frameshift |
| E672del | deletion of 3 bp between | 13 | deletion of Glu |
| | 2145-2148 | | at 672 |
| G673X | G to T at 2149 | 13 | Gly to Stop at 673 |
| W679X | G to A at 2168 | 13 | Trp to stop at 679 |
| 2176insC | insertion of C after 2176 | 13 | Frameshift |
| K683R | A to G at 2180 | 13 | Lys to Arg at 683 |
| 2183AA −> G | A to G at 2183 and | 13 | Frameshift |
| | deletion of A at 2184 | | |
| 2183delAA | deletion of AA at 2183 | 13 | Frameshift |
| 2184delA | deletion of A at 2184 | 13 | frameshift |
| 2184insG | inserion of G after 2184 | 13 | Frameshift |
| 2184insA | insertion of A after 2184 | 13 | Frameshift |
| 2185insC | insertion of C at 2185 | 13 | Frameshift |
| Q685X | C to T at 2185 | 13 | Gln to Stop at 685 |
| E692X | G to T at 2206 | 13 | Glu to Stop at 692 |
| F693L(CTT) | T to C at 2209 | 13 | Phe to Leu at 693 |
| F693L(TTG) | T to G at 2211 | 13 | Phe to Leu at 693 |
| 2215insG | insertion of G at 2215 | 13 | Frameshift |
| K698R | A to G 2225 | 13 | Lys to Arg at 698 |
| R709X | C to T at 2257 | 13 | Arg to Stop at 709 |
| K710X | A to T at 2260 | 13 | Lys to Stop at 710 |
| K716X | AA to GT at 2277 and | 13 | Lys to Stop at 716 |
| | 2278 | | |
| L719X | T to A at 2288 | 13 | Leu to Stop at 719 |
| Q720X | C to T at 2290 | 13 | Gln to stop codon |
| | | | at 720 |
| E725K | G to A at 2305 | 13 | Glu to Lys at 725 |
| 2307insA | insertion of A after 2307 | 13 | Frameshift |
| E730X | G to T at 2320 | 13 | Glu to Stop at 730 |
| L732X | T to G at 2327 | 13 | Leu to Stop at 732 |
| 2335delA | deletion of A at 2335 | 13 | Frameshift |
| R735K | G to A at 2336 | 13 | Arg to Lys at 735 |
| 2347delG | deletion of G at 2347 | 13 | Frameshift |
| 2372del8 | deletion of 8 bp from | 13 | Frameshift |
| | 2372 | | |
| P750L | C to T at 2381 | 13 | Pro to Leu at 750 |
| V754M | G to A at 2392 | 13 | Val to Met at 754 |
| T760M | C to T at 2411 | 13 | Thr to Met at 760 |
| R764X | C to T at 2422 | 13 | Arg to Stop at 764 |
| 2423delG | deletion of G at 2423 | 13 | Frameshift |
| R766M | G to T at 2429 | 13 | Arg to Met at 766 |
| 2456delAC | deletion of AC at 2456 | 13 | Frameshift |
| S776X | C to G at 2459 | 13 | Ser to Stop at 776 |

TABLE 11
CFTR mutations that may be detected in amplified product
using as the primer pair SEQ ID NO: 11 and 12.

| Name | Nucleotide_change | Exon | Consequence |
|---|---|---|---|
| T908N | C to A at 2788 | 14b | Thr to Asn at 908 |
| 2789 + 2insA | insertion of A after | intron 14b | mRNA splicing |
| | 2789 + 2 | | defect? (CAVD) |
| 2789 + 3delG | deletion of G at | intron 14b | mRNA splicing |
| | 2789 + 3 | | defect |
| 2789 + 5G −> A | G to A at 2789 + 5 | intron 14b | mRNA splicing |
| | | | defect |

TABLE 12
CFTR mutations that may be detected in amplified product
using as the primer pair SEQ ID NO: 25 and 26.

| Name | Nucleotide_change | Exon | Consequence |
|---|---|---|---|
| 3100insA | insertion of A | 16 | Frameshift |
| | after 3100 | | |
| I991V | A to G at 3103 | 16 | Ile to Val at 991 |
| D993Y | G to T at 3109 | 16 | Asp to Tyr at 993 |
| F994C | T to G at 3113 | 16 | Phe to Cys at 994 |
| 3120G −> A | G to A at 3120 | 16 | mRNA splicing defect |
| 3120 + | G to A at 3120 + 1 | intron 16 | mRNA splicing defect |
| 1G −> A | | | |

TABLE 13
CFTR mutations that may be detected in amplified product
using as the primer pair SEQ ID NO: 29 and 30.

| Name | Nucleotide_change | Exon | Consequence |
|---|---|---|---|
| 3601 − 20T −> C | T to C at 3601 − 20 | intron 18 | mRNA splicing |
| | | | mutant? |
| 3601 − 17T −> C | T to C at 3601 − 17 | intron 18 | mRNA splicing |
| | | | defect? |
| 3601 − 2A −> G | A to G at 3601 − 2 | intron 18 | mRNA splicing |
| | | | defect |
| R1158X | C to T at 3604 | 19 | Arg to Stop at 1158 |
| S1159P | T to C at 3607 | 19 | Ser to Pro at 115p |
| S1159F | C to T at 3608 | 19 | Ser to Phe at 1159 |
| R1162X | C to T at 3616 | 19 | Arg to Stop at 1162 |
| 3622insT | insertion of T | 19 | Frameshift |
| | after 3622 | | |
| D1168G | A to G at 3635 | 19 | Asp to Gly at 1168 |
| 3659delC | deletion of C | 19 | Frameshift |
| | at 3659 | | |
| K1177X | A to T at 3661 | 19 | Lys to Stp at 3661 |
| | | | (premature termina- |
| | | | tion) |
| K1177R | A to G at 3662 | 19 | Lys to Arg at 1177 |
| 3662delA | deletion of A | 19 | Frameshift |
| | at 3662 | | |
| 3667del4 | deletion of 4 | 19 | Frameshift |
| | bp from 3667 | | |
| 3667ins4 | insertion of TCAA | 19 | Frameshift |
| | after 3667 | | |
| 3670delA | deletion of A | 19 | Frameshift |
| | at 3670 | | |
| Y1182X | C to G at 3678 | 19 | Tyr to Stop at 1182 |
| Q1186X | C to T at 3688 | 19 | Gln to Stop codon |
| | | | at 1186 |
| 3696G/A | G to A at 3696 | 18 | No change to Ser |
| | | | at 1188 |
| V1190P | T to A at 3701 | 19 | Val to Pro at 1190 |
| S1196T | C or Q at 3719 | 19 | Ser-Top at 1196 |
| S1196X | C to G at 3719 | 19 | Ser to Stop at 1196 |
| 3724delG | deletion of G | 19 | Frameshift |
| | at 3724 | | |
| 3732delA | deletion of A | 19 | frameshift and Lys |
| | at 3732 and A | | to Glu at 1200 |
| | to G at 3730 | | |
| 3737delA | deletion of A | 19 | Frameshift |
| | at 3737 | | |
| W1204X | G to A at 3743 | 19 | Trp to Stop at 1204 |
| S1206X | C to G at 3749 | 19 | Ser to Stop at 1206 |
| 3750delAG | deletion of AG | 19 | Frameshift |
| | from 3750 | | |
| 3755delG | deletion of G | 19 | Frameshift |
| | between 3751 | | |
| | and 3755 | | |
| M1210I | G to A at 3762 | 19 | Met to Ile at 1210 |
| V1212I | G to A at 3766 | 19 | Val to Ile at 1212 |

TABLE 14
CFTR mutations that may be detected in amplified product
using as the primer pair SEQ ID NO: 5 and 6.

| Name | Nucleotide_change | Exon | Consequence |
|---|---|---|---|
| 3849 + | C to T in a 6.2 kb EcoRI | intron | creation of |
| 10 kb | fragment 10 kb from 19 | 19 | splice acceptor |
| C −> T | | | site |

TABLE 15
CFTR mutations that may be detected in amplified product
using as the primer pair SEQ ID NO: 17 and 18.

| Name | Nucleotide_change | Exon | Consequence |
|---|---|---|---|
| T1252P | A to C at 3886 | 20 | Thr to Pro at 1252 |
| L1254X | T to G at 3893 | 20 | Leu to Stop at 1254 |
| S1255P | T to C at 3895 | 20 | Ser to Pro at 1255 |
| S1255L | C to T at 3896 | 20 | Ser to Leu at 1255 |
| S1255X | C to A at 3896 and A to | 20 | Ser to Stop at 1255 |
| | G at 3739 in exon 19 | | and Ile to Val at |
| | | | 1203 |
| 3898insC | insertion of C | 20 | Frameshift |
| | after 3898 | | |
| F1257L | T to G at 3903 | 20 | Phe to Leu at 1257 |
| 3905insT | insertion of T | 20 | Frameshift |
| | after 3905 | | |
| 3906insG | insertion of G | 20 | Frameshift |
| | after 3906 | | |
| [delta]L1260 | deletion of ACT from | 20 | deletion of Leu at |
| | either 3909 or 3912 | | 1260 or 1261 |
| 3922del10 −> C | deletion of 10 bp from | 20 | deletion of Glu1264 |
| | 3922 and replacement | | to Glu1266 |
| | with 3921 | | |
| I1269N | T to A at 3938 | 20 | Ile to Asn at 1269 |
| D1270N | G to A at 3940 | 20 | Asp to Asn at 1270 |
| 3944delGT | deletion of GT from | 20 | Frameshift |
| | 3944 | | |
| W1274X | G to A at 3954 | 20 | Trp to Stop at 1274 |
| Q1281X | C to T at 3973 | 20 | Gln to Stop at 1281 |
| W1282R | T to C at 3976 | 20 | Trp to Arg at 1282 |
| W1282G | T to G at 3976 | 20 | Trp to Gly at 1282 |
| W1282X | G to A at 3978 | 20 | Trp to Stop at 1282 |
| W1282C | G to T at 3978 | 20 | Trp to Cys at 1282 |
| R1283M | G to T at 3980 | 20 | Arg to Met at 1283 |
| R1283K | G to A at 3980 | 20 | Arg to Lys at 1283 |
| F1286S | T to C at 3989 | 20 | Phe to Ser at 1286 |

TABLE 16
CFTR mutations that may be detected in amplified product
using as the primer pair SEQ ID NO: 13 and 14.

| Name | Nucleotide_change | Exon | Consequence |
|---|---|---|---|
| T1299I | C to T at 4028 | 21 | Thr to Ile at 1299 |
| F1300L | T to C at 4030 | 21 | Phe to Leu at 1300 |
| N1303H | A to C at 4039 | 21 | Asn to His at 1303 |
| N1303I | A to T at 4040 | 21 | Asn to Ile at 1303 |
| 4040delA | deletion of A at 4040 | 21 | Frameshift |
| N1303K | C to G at 4041 | 21 | Asn to Lys at 1303 |
| D1305E | T to A at 4047 | 21 | Asp to Glu at 1305 |
| 4048insCC | insertion of CC after | 21 | Frameshift |
| | 4048 | | |
| Y1307X | T to A at 4053 | 21 | Tyr to Stop at 1307 |
| E1308X | G to T at 4054 | 21 | Glu to Stop at 1308 |

CF mutations including those known under symbols: 2789+5G>A; 711+1G>T; W1282X; 3120+1G>A; d1507; dF508; (F508C, 1507V, 1506V); N1303K; G542X, G551D, R553X, R560T, 1717-1G>A: R334W, R347P, 1078delT; R117H, I148T, 621+1G>T; G85E; R1162X, 3659delC; 2184delA; A455E, (5T, 7T, 9T); 3849+10kbC>T; and 1898+1G>A, are described in U.S. patent application Ser. No. 396,894, filed Apr. 22, 1989, application Ser. No. 399,945, filed Aug. 29, 1989, application Ser. No. 401,609 filed Aug. 31, 1989. and U.S. Pat. Nos. 6,011,588 and 5,981,178, which are hereby incorporated by reference in their entirety. Any and all of these mutations can be detected using nucleic acid amplified with the invention primers as described herein.

CF mutations in the amplified nucleic acid may be identified in any of a variety of ways well known to those of ordinary skill in the art. For example, if an amplification product is of a characteristic size, the product may be detected by examination of an electrophoretic gel for a band at a precise location. In another embodiment, probe molecules that hybridize to the mutant or wildtype CF sequences can be used for detecting such sequences in the amplified product by solution phase or, more preferably, solid phase hybridization. Solid phase hybridization can be achieved, for example, by attaching the CF probes to a microchip. Probes for detecting CF mutant sequences are well known in the art. See Wall et al. “A 31-mutation assay for cystic fibrosis testing in the clinical molecular diagnostics laboratory,” Human Mutation, 1995;5(4):333-8, which specifies probes for CF mutations ΔF508 (exon 1), G542X (exon 11), G551D (exon 11), R117H (exon 4), W1282X (exon 20), N1303K (exon 21), 3905insT (exon 20), 3849+10Kb (intron 19), G85E (exon 3), R334W (exon 7), A455E (exon 9), 1898+1 (exon 12), 2184delA (exon 13), 711+1 (exon 5), 2789+5 (exon 14b), Y1092x exon 17b), ΔI507 (exon 10), S549R(T-G) (exon 11), 621+1 (exon 4), R1162X (exon 19), 1717-1 (exon 11), 3659delC (exon 19), R560T (exon 11), 3849+4(A-G) (exon 19), Y122X (exon 4), R553X (exon11), R347P (exon 7), R347H (exon 7), Q493X (exon 10), V520F (exon 10), and S549N (exon 11). Probes for additional CF mutations include those shown in Table 17.

TABLE 17

Probes for Detection of CF mutations

| | | |
|---|---|---|
| CF Mutation | Name | Sequence |

| | | |
|---|---|---|
| I148T | SNP1 | 5′-CCATTTTTGGCCTTCATCACA-3′ |
| | | (SEQ ID NO: 33) |
| 2184delA | SNP3 | 5′-GATCGATCTGTCTCCTGGACAGAAAC |
| | | AAAAAA-3′ |
| | | (SEQ ID NO: 34) |
| D1270N | SNP5 | 5′-GACTGATCGATCGTTATTGAATCCCA |
| | | AGACACACCAT-3′ |
| | | (SEQ ID NO: 35) |
| 3120 + 1 G -> A | SNP6 | 5′-GACTGATCGATCGATCCCTCTTACCA |
| | | TATTTGACTTCATCCAG-3′ |
| | | (SEQ ID NO: 36) |

CF probes for detecting mutations as described herein may be attached to a solid phase in the form of an array as is well known in the art (see, U.S. Pat. Nos. 6,403,320 and 6,406,844). For example, the full complement of 24 probes for CF mutations with additional control probes (30 in total) can be conjugated to a silicon chip essentially as described by Jenison et al., Biosens Bioelectron. 16(9-12):757-63 (2001) (see also U.S. Pat. Nos. 6,355,429 and 5,955,377). Amplicons that hybridized to particular probes on the chip can be identified by transformation into molecular thin films. This can be achieved by contacting the chip with an anti-biotin antibody or streptavidin conjugated to an enzyme such as horseradish peroxidase. Following binding of the antibody(or streptavidin)-enzyme conjugate to the chip, and washing away excess unbound conjugate, a substrate can be added such as tetramethylbenzidine (TMB) {3,3′,5,5′Tetramethylbenzidine} to achieve localized deposition (at the site of bound antibody) of a chemical precipitate as a thin film on the surface of the chip. Other enzyme/substrate systems that can be used are well known in the art and include, for example, the enzyme alkaline phosphatase and 5-bromo-4-chloro-3-indolyl phosphate as the substrate. The presence of deposited substrate on the chip at the locations in the array where probes are attached can be read by an optical scanner. U.S. Pat. Nos. 6,355,429 and 5,955,377, which are hereby incorporated by reference in their entirety including all charts and drawings, describe preferred devices for performing the methods of the present invention and their preparation, and describes methods for using them.

The binding of amplified nucleic acid to the probes on the solid phase following hybridization may be measured by methods well known in the art including, for example, optical detection methods described in U.S. Pat. No. 6,355,429. In preferred embodiments, an array platform (see, e.g., U.S. Pat. No. 6,288,220) can be used to perform the methods of the present invention, so that multiple mutant DNA sequences can be screened simultaneously. The array is preferably made of silicon, but can be other substances such as glass, metals, or other suitable material, to which one or more capture probes are attached. In preferred embodiments, at least one capture probe for each possible amplified product is attached to an array. Preferably an array contains 10, more preferably 20, even more preferably 30, and most preferably at least 60 different capture probes covalently attached to the array, each capture probe hybridizing to a different CF mutant sequence. Nucleic acid probes useful as positive and negative controls also may be included on the solid phase or used as controls for solution phase hybridization.

In still another approach, wildtype or mutant CF sequence in amplified DNA may be detected by direct sequence analysis of the amplified products. A variety of methods can be used for direct sequence analysis as is well known in the art. See, e.g., The PCR Technique: DNA Sequencing (eds. James Ellingboe and Ulf Gyllensten) Biotechniques Press, 1992; see also “SCAIP” (single condition amplification/internal primer) sequencing, by Flanigan et al. Am J Hum Genet. 2003 April;72(4):931-9. Epub Mar. 11, 2003.

In yet another approach for detecting wildtype or mutant CF sequences in amplified DNA is single nucleotide primer extension or “SNuPE.” SNUPE can be performed as described in U.S. Pat. No. 5,888,819 to Goelet et al., U.S. Pat. No. 5,846,710 to Bajaj, Piggee, C. et al. Journal of Chromatography A 781 (1997), p. 367-375 (“Capillary Electrophoresis for the Detection of Known Point Mutations by Single-Nucleotide Primer Extension and Laser-Induced Fluorescence Detection”); Hoogendoom, B. et al., Human Genetics (1999) 104:89-93, (“Genotyping Single Nucleotide Polymorphism by Primer Extension and High Performance Liquid Chromatography”); and U.S. Pat. No. 5,885,775 to Haff et al. (analysis of single nucleotide polymorphism analysis by mass spectrometry). In SNuPE, one may use as primers such as those specified in Table 17.

Still another approach for detecting wildtype or mutant CF sequences in amplified DNA is oligonucleotide ligation assay or “OLA”. The OLA uses two oligonucleotides which are designed to be capable of hybridizing to abutting sequences of a single strand of a target molecules. One of the oligonucleotides is biotinylated, and the other is detectably labeled. If the precise complementary sequence is found in a target molecule, the oligonucleotides will hybridize such that their termini abut, and create a ligation substrate that can be captured and detected. See e.g., Nickerson et al. (1990) Proc. Natl. Acad. Sci. U.S.A. 87:8923-8927, Landegren, U. et al. (1988) Science 241:1077-1080 and U.S. Pat. No. 4,998,617.

These above approaches for detecting wildtype or mutant CF sequence in the amplified nucleic acid is not meant to be limiting, and those of skill in the art will understand that numerous methods are known for determining the presence or absence of a particular nucleic acid amplification product.

In another aspect the present invention provides kits for one of the methods described herein. In various embodiments, the kits contain one or more of the invention primers in an amount suitable for amplifying a specified CFTR sequence from at least one nucleic acid containing sample. The kit optionally contain buffers, enzymes, and reagents for amplifying the CFTR nucleic acid via primer-directed amplification. The kit also may include one or more devices for detecting the presence or absence of particular mutant CF sequences in the amplified nucleic acid. Such devices may include one or more probes that hybridize to a mutant CF nucleic acid sequence, which preferably is attached to a bio-chip device, such as any of those described in U.S. Pat. No. 6,355,429. The bio-chip device optionally has at least one capture probe attached to a surface on the bio-chip that hybridizes to a mutant CF sequence. In preferred embodiments the bio-chip contains multiple probes, and most preferably contains at least one probe for a mutant CF sequence which, if present, would be amplified by a set of flanking primers. For example, if five pairs of flanking primers are used for amplification, the device would contain at least one CF mutant probe for each amplified product, or at least five probes. The kit also preferably contains instructions for using the components of the kit.

The following examples serve to illustrate the present invention. These examples are in no way intended to limit the scope of the invention.

### EXAMPLE 1

### Detection of CF Mutations from Whole Blood

A. Extraction of DNA

Suitable samples may include fresh tissue, e.g., obtained from clinical swabs from a region where cells are collected by soft abrasion (e.g., buccal, cervical, vaginal, etc. surfaces) or biopsy specimens; cells obtained by amniocentesis or chorionic villus sampling; cultured cells, or blood cells; or may include fixed or frozen tissues. The following example describes preparation of nucleic acids from blood.

50 μL of whole blood was mixed with 0.5 ml of TE (10 mM Tris HCl, 1 mM EDTA, pH 7.5) in a 1.5 mL microfuge tube. The sample was spun for 10 seconds at 13,000×g. The pellet was resuspended in 0.1 mL of TE buffer with vortexing, and pelleted again. This procedure was repeated twice more, and then the final cell pellet was resuspended in 100 μl of K buffer 50 mM KCl, 10 mM Tris HCl, 2.5 mM MgCl₂, 0.5% Tween 20, 100 μg/mL proteinase K, pH 8.3) and incubated 45 minutes at 56° C., then 10 minutes at 95° C. to inactivate the protease.

B. Amplification from DNA

Individual amplifications were prepared in a volume of 13.5 μl, which was added to 96 well microtiter plates. Each amplification volume contained 2 μl of the DNA sample (generally 10-100 ng of DNA), 11.5 μl of PCR-Enzyme Mix (PCR-Enzyme mix stock was prepared with 11.3 μl master mix, 0.25 μl MgCl₂ (from 25 mM stock), and 0.2 μl of FasStar Taq (source for last two reagents was Roche Applied science, Cat. No. 2 032 937). Master mix contained 5′ biotinylated primers, Roche PCR buffer with MgCl₂, Roche GC rich solution (cat. No. 2 032 937), bovine serum albumin (BSA) (New England BioLabs, Cat no. B9001B), and NTPs (Amersham Biosciences, Cat no. 27-2032-01).

The final concentration in the PCR for MgCl₂ was 2.859 mM, for BSA was 0.725 μg/μl, and for each dNTP was 0.362 mM. Primer final concentrations of biotinylated primers were 0.29 μM for each of SEQ ID NOs: 9, 10, 13 and 14 (exon 12 and 21), 0.145 μM for each of SEQ ID NOs: 3-6 (exons 4 and i19), 0.091 μM for each of SEQ ID NOs: 7, 8, 15, 16, and 29-32 (exons 19, 7, i5 and 11), 0.072 μM for each of SEQ ID NOs: 11, 12, 19 and 20 (exon 3 and 14), 0.060 μM for each of SEQ ID NOs: 17, 18 and 23-28 (exons 16, 20, 13 and 10), and 0.036 μM for each of SEQ ID NOs: 21 and 22, (exon 9).

PCR was conducted using the following temperature profile: step 1: 96° C. for 15 minutes; step 2: 94° C. for 15 seconds; step 3: decrease at 0.5° C./second to 56° C.; step 4: 56° C. for 20 seconds; step 5: increase at 0.3° C./second to 72° C., step 6: 72° C. for 30 seconds; step 7: increase 0.5° C. up to 94° C.; step 8: repeat steps 2 to 7 thirty three times; step 9: 72° C. for 5 minutes; step 10: 4° C. hold (to stop the reaction).

C. Detection of CF Amplicons

The presence of CF sequences in the amplicons was determined by hybridizing the amplified product to a solid phase strip containing an array of 50 probes specific for CF mutations and CF wildtype sequence (LINEAR ARRAY CF GOLD 1.0™, Roche Diagnostics) in accordance with the manufacturer's instructions. Detection of hybridized amplicons was by streptavidin-HRP conjugate and development using the TMB as substrate.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A substantially purified nucleic acid sample comprising one or more nucleic acids having sequences selected from the group consisting of:

| | |
|---|---|
| 5′-GCGGTCCCAAAAGGGTCAGTTGTAGGAAGTCACCAAAG-3′, | (SEQ ID NO: 3) |
| 5′-GCGGTCCCAAAAGGGTCAGTCGATACAGAATATATGTGCC-3′, | (SEQ ID NO: 4) |
| 5′-GCGGTCCCAAAAGGGTCAGTGAATCATTCAGTGGGTATAAGCAG-3′, | (SEQ ID NO: 5) |
| 5′-GCGGTCCCAAAAGGGTCAGTCTTCAATGCACCTCCTCCC-3′, | (SEQ ID NO: 6) |
| 5′-GCGGTCCCAAAAGGGTCAGTAGATACTTCAATAGCTCAGCC-3′, | (SEQ ID NO: 7) |
| 5′-GCGGTCCCAAAAGGGTCAGTGGTACATTACCTGTATTTTGTTT-3′, | (SEQ ID NO: 8) |
| 5′-GCGGTCCCAAAAGGGTCAGTGTGAATCGATGTGGTGACCA-3′, | (SEQ ID NO: 9) |
| 5′-GCGGTCCCAAAAGGGTCAGTCTGGTTTAGCATGAGGCGGT-3′, | (SEQ ID NO: 10) |
| 5′-GCGGTCCCAAAAGGGTCAGTTTGGTTGTGCTGTGGCTCCT-3′, | (SEQ ID NO: 11) |
| 5′-GCGGTCCCAAAAGGGTCAGTACAATACATACAAACATAGTGG-3′, | (SEQ ID NO: 12) |
| 5′-GCGGTCCCAAAAGGGTCAGTGAAAGTATTTATTTTTTCTGGAAC-3′, | (SEQ ID NO: 13) |
| 5′-GCGGTCCCAAAAGGGTCAGTGTGTGTAGAATGATGTCAGCTAT-3′, | (SEQ ID NO: 14) |
| 5′-GCGGTCCCAAAAGGGTCAGTCAGATTGAGCATACTAAAAGTG-3′, | (SEQ ID NO: 15) |
| 5′-GCGGTCCCAAAAGGGTCAGTTACATGAATGACATTTACAGCA-3′, | (SEQ ID NO: 16) |
| 5′-GCGGTCCCAAAAGGGTCAGTAAGAACTGGATCAGGGAAGA-3′, | (SEQ ID NO: 17) |
| 5′-GCGGTCCCAAAAGGGTCAGTTCCTTTTGCTCACCTGTGGT-3′, | (SEQ ID NO: 18) |
| 5′-GCGGTCCCAAAAGGGTCAGTGGTCCCACTTTTTATTCTTTTGC-3′, | (SEQ ID NO: 19) |
| 5′-GCGGTCCCAAAAGGGTCAGTTGGTTTCTTAGTGTTTGGAGTTG-3′, | (SEQ ID NO: 20) |
| 5′-GCGGTCCCAAAAGGGTCAGTTGGATCATGGGCCATGTGC-3′, | (SEQ ID NO: 21) |
| 5′-GCGGTCCCAAAAGGGTCAGTACTACCTTGCCTGCTCCAGTGG-3′, | (SEQ ID NO: 22) |
| 5′-GCGGTCCCAAAAGGGTCAGTAGGTAGCAGCTATTTTTATGG-3′, | (SEQ ID NO: 23) |
| 5′-GCGGTCCCAAAAGGGTCAGTTAAGGGAGTCTTTTGCACAA-3′, | (SEQ ID NO: 24) |
| 5′-GCGGTCCCAAAAGGGTCAGTGCAATTTTGGATGACCTTC-3′, | (SEQ ID NO: 25) |
| 5′-GCGGTCCCAAAAGGGTCAGTTAGACAGGACTTCAACCCTC-3′, | (SEQ ID NO: 26) |
| 5′-GCGGTCCCAAAAGGGTCAGTGGTGATTATGGGAGAACTGG-3′, | (SEQ ID NO: 27) |
| 5′-GCGGTCCCAAAAGGGTCAGTATGCTTTGATGACGCTTC-3′, | (SEQ ID NO: 28) |
| 5′-GCGGTCCCAAAAGGGTCAGTTTCATTGAAAAGCCCGAC-3′, | (SEQ ID NO: 29) |
| 5′-GCGGTCCCAAAAGGGTCAGTCACCTTCTGTGTATTTTGCTG-3′, | (SEQ ID NO: 30) |
| 5′-GCGGTCCCAAAAGGGTCAGTAAGTATTGGACAACTTGTTAGTCTC-3′, | (SEQ ID NO: 31) |
| 5′-GCGGTCCCAAAAGGGTCAGTCGCCTTTCCAGTTGTATAATTT-3′, | (SEQ ID NO: 32) |

or a complementary nucleic acid sequence thereof.

2. The substantially purified nucleic acid of claim 1 wherein said composition is labeled with a detectable label.

3. The substantially purified nucleic acid of claim 1 wherein said detectable label is biotin.

4. A method of amplifying a nucleic acid sequence, comprising,
contacting a nucleic acid containing sample with reagents suitable for nucleic acid amplification including one or more pairs of primers flanking one or more predetermined nucleic acid sequences that are correlated with cystic fibrosis, and
amplifying said one or more predetermined nucleic acid sequences, if present, wherein said primers are one or more pairs of nucleic acids selected from the group consisting of:

| | |
|---|---|
| 5′-GCGGTCCCAAAAGGGTCAGTTGTAGGAAGTCACCAAAG-3′, | (SEQ ID NO: 3) |
| 5′-GCGGTCCCAAAAGGGTCAGTCGATACAGAATATATGTGCC-3′, | (SEQ ID NO: 4) |
| 5′-GCGGTCCCAAAAGGGTCAGTGAATCATTCAGTGGGTATAAGCAG-3′, | (SEQ ID NO: 5) |
| 5′-GCGGTCCCAAAAGGGTCAGTCTTCAATGCACCTCCTCCC-3′, | (SEQ ID NO: 6) |
| 5′-GCGGTCCCAAAAGGGTCAGTAGATACTTCAATAGCTCAGCC-3′, | (SEQ ID NO: 7) |
| 5′-GCGGTCCCAAAAGGGTCAGTGGTACATTACCTGTATTTTGTTT-3′, | (SEQ ID NO: 8) |
| 5′-GCGGTCCCAAAAGGGTCAGTGTGAATCGATGTGGTGACCA-3′, | (SEQ ID NO: 9) |
| 5′-GCGGTCCCAAAAGGGTCAGTCTGGTTTAGCATGAGGCGGT-3′, | (SEQ ID NO: 10) |
| 5′-GCGGTCCCAAAAGGGTCAGTTTGGTTGTGCTGTGGCTCCT-3′, | (SEQ ID NO: 11) |
| 5′-GCGGTCCCAAAAGGGTCAGTACAATACATACAAACATAGTGG-3′, | (SEQ ID NO: 12) |
| 5′-GCGGTCCCAAAAGGGTCAGTGAAAGTATTTATTTTTTCTGGAAC-3′, | (SEQ ID NO: 13) |
| 5′-GCGGTCCCAAAAGGGTCAGTGTGTGTAGAATGATGTCAGCTAT-3′, | (SEQ ID NO: 14) |
| 5′-GCGGTCCCAAAAGGGTCAGTCAGATTGAGCATACTAAAAGTG-3′, | (SEQ ID NO: 15) |
| 5′-GCGGTCCCAAAAGGGTCAGTTACATGAATGACATTTACAGCA-3′, | (SEQ ID NO: 16) |
| 5′-GCGGTCCCAAAAGGGTCAGTAAGAACTGGATCAGGGAAGA-3′, | (SEQ ID NO: 17) |
| 5′-GCGGTCCCAAAAGGGTCAGTTCCTTTTGCTCACCTGTGGT-3′, | (SEQ ID NO: 18) |
| 5′-GCGGTCCCAAAAGGGTCAGTGGTCCCACTTTTTATTCTTTTGC-3′, | (SEQ ID NO: 19) |
| 5′-GCGGTCCCAAAAGGGTCAGTTGGTTTCTTAGTGTTTGGAGTTG-3′, | (SEQ ID NO: 20) |
| 5′-GCGGTCCCAAAAGGGTCAGTTGGATCATGGGCCATGTGC-3′, | (SEQ ID NO: 21) |
| 5′-GCGGTCCCAAAAGGGTCAGTACTACCTTGCCTGCTCCAGTGG-3′, | (SEQ ID NO: 22) |
| 5′-GCGGTCCCAAAAGGGTCAGTAGGTAGCAGCTATTTTTATGG-3′, | (SEQ ID NO: 23) |
| 5′-GCGGTCCCAAAAGGGTCAGTTAAGGGAGTCTTTTGCACAA-3′, | (SEQ ID NO: 24) |
| 5′-GCGGTCCCAAAAGGGTCAGTGCAATTTTGGATGACCTTC-3′, | (SEQ ID NO: 25) |
| 5′-GCGGTCCCAAAAGGGTCAGTTAGACAGGACTTCAACCCTC-3′, | (SEQ ID NO: 26) |
| 5′-GCGGTCCCAAAAGGGTCAGTGGTGATTATGGGAGAACTGG-3′, | (SEQ ID NO: 27) |
| 5′-GCGGTCCCAAAAGGGTCAGTATGCTTTGATGACGCTTC-3′, | (SEQ ID NO: 28) |
| 5′-GCGGTCCCAAAAGGGTCAGTTTCATTGAAAAGCCCGAC-3′, | (SEQ ID NO: 29) |
| 5′-GCGGTCCCAAAAGGGTCAGTCACCTTCTGTGTATTTTGCTG-3′, | (SEQ ID NO: 30) |
| 5′-GCGGTCCCAAAAGGGTCAGTAAGTATTGGACAACTTGTTAGTCTC-3′, | (SEQ ID NO: 31) |
| 5′-GCGGTCCCAAAAGGGTCAGTCGCCTTTCCAGTTGTATAATTT-3′. | (SEQ ID NO: 32) |

5. The method of claim 4, wherein said one or more pairs of nucleic acid primers is five pairs of nucleic acid primers.

6. The method of claim 4, wherein said one or more pairs of nucleic acid primers is ten pairs of nucleic acid primers.

7. The method of claim 4, wherein said one or more pairs of nucleic acid primers is fifteen pairs of nucleic acid primers.

8. The method of claim 7, wherein said primer sets are added in the following ratios determined as the moles of primers for exon 12 and 21 (SEQ ID NO: 9, 10, 13 and 14) to the moles of each other primer sets, the ratio being about 2 for exons 4 and i19 (SEQ ID NOs; 3-6), about 3.2 for exons 19, 7, 11 and i5 (SEQ ID NOs; 7, 8, 15, 16, and 29-32), about 4 for exons 3 and 14 (SEQ ID NOs; 11, 12, 19, 20), about 4.8 for exons 16, 20, 13 and 10 (SEQ ID NOs; 17, 18, 23 and 28), and about 8 for exon 9 (SEQ ID NOs; 22 and 21).

9. The method of claim 4, wherein said amplifying is by the polymerase chain reaction.

10. A method of determining the presence or absence of one or nucleic acid sequences correlated with cystic fibrosis in a nucleic acid containing sample, comprising:
contacting said sample with reagents suitable for nucleic acid amplification including one or more pairs of nucleic acid primers flanking one or more predetermined nucleic acid sequences that are correlated with cystic fibrosis,
amplifying said predetermined nucleic acid sequence(s), if present, to provide an amplified sample; and
identifying the presence or absence of said one or more predetermined sequences in said amplified sample, whereby the presence or absence of said one or more nucleic acid sequences correlated with cystic fibrosis is determined;
wherein said pairs of nucleic acid primers are selected from the group consisting of:

| | |
|---|---|
| 5′-GCGGTCCCAAAAGGGTCAGTTGTAGGAAGTCACCAAAG-3′ | (SEQ ID NO: 3) |
| and | |
| 5′-GCGGTCCCAAAAGGGTCAGTCGATACAGAATATATGTGCC-3′, | (SEQ ID NO: 4) |
| 5′-GCGGTCCCAAAAGGGTCAGTGAATCATTCAGTGGGTATAAGCAG-3′ | (SEQ ID NO: 5) |
| and | |
| 5′-GCGGTCCCAAAAGGGTCAGTCTTCAATGCACCTCCTCCC-3′, | (SEQ ID NO: 6) |
| 5′-GCGGTCCCAAAAGGGTCAGTAGATACTTCAATAGCTCAGCC-3′ | (SEQ ID NO: 7) |
| and | |
| 5′-GCGGTCCCAAAAGGGTCAGTGGTACATTACCTGTATTTTGTTT-3′, | (SEQ ID NO: 8) |
| 5′-GCGGTCCCAAAAGGGTCAGTGTGAATCGATGTGGTGACCA-3′ | (SEQ ID NO: 9) |
| and | |
| 5′-GCGGTCCCAAAAGGGTCAGTCTGGTTTAGCATGAGGCGGT-3′, | (SEQ ID NO: 10) |
| 5′-GCGGTCCCAAAAGGGTCAGTTTGGTTGTGCTGTGGCTCCT-3′ | (SEQ ID NO: 11) |
| and | |
| 5′-GCGGTCCCAAAAGGGTCAGTACAATACATACAAACATAGTGG-3′, | (SEQ ID NO: 12) |
| 5′-GCGGTCCCAAAAGGGTCAGTGAAAGTATTTATTTTTTCTGGAAC-3′ | (SEQ ID NO: 13) |
| and | |
| 5′-GCGGTCCCAAAAGGGTCAGTGTGTGTAGAATGATGTCAGCTAT-3′, | (SEQ ID NO: 14) |
| 5′-GCGGTCCCAAAAGGGTCAGTCAGATTGAGCATACTAAAAGTG-3′ | (SEQ ID NO: 15) |
| and | |
| 5′-GCGGTCCCAAAAGGGTCAGTTACATGAATGACATTTACAGCA-3′, | (SEQ ID NO: 16) |
| 5′-GCGGTCCCAAAAGGGTCAGTAAGAACTGGATCAGGGAAGA-3′ | (SEQ ID NO: 17) |
| and | |
| 5′-GCGGTCCCAAAAGGGTCAGTTCCTTTTGCTCACCTGTGGT-3′, | (SEQ ID NO: 18) |
| 5′-GCGGTCCCAAAAGGGTCAGTGGTCCCACTTTTTATTCTTTTGC-3′ | (SEQ ID NO: 19) |
| and | |
| 5′-GCGGTCCCAAAAGGGTCAGTTGGTTTCTTAGTGTTTGGAGTTG-3′, | (SEQ ID NO: 20) |
| 5′-GCGGTCCCAAAAGGGTCAGTTGGATCATGGGCCATGTGC-3′ | (SEQ ID NO: 21) |
| and | |
| 5′-GCGGTCCCAAAAGGGTCAGTACTACCTTGCCTGCTCCAGTGG-3′, | (SEQ ID NO: 22) |
| 5′-GCGGTCCCAAAAGGGTCAGTAGGTAGCAGCTATTTTTATGG-3′ | (SEQ ID NO: 23) |
| and | |
| 5′-GCGGTCCCAAAAGGGTCAGTTAAGGGAGTCTTTTGCACAA-3′, | (SEQ ID NO: 24) |
| 5′-GCGGTCCCAAAAGGGTCAGTGCAATTTTGGATGACCTTC-3′ | (SEQ ID NO: 25) |
| and | |
| 5′-GCGGTCCCAAAAGGGTCAGTTAGACAGGACTTCAACCCTC-3′, | (SEQ ID NO: 26) |
| 5′-GCGGTCCCAAAAGGGTCAGTGGTGATTATGGGAGAACTGG-3′ | (SEQ ID NO: 27) |
| and | |
| 5′-GCGGTCCCAAAAGGGTCAGTATGCTTTGATGACGCTTC-3′, | (SEQ ID NO: 28) |
| 5′-GCGGTCCCAAAAGGGTCAGTTTCATTGAAAAGCCCGAC-3′ | (SEQ ID NO: 29) |
| and | |
| 5′-GCGGTCCCAAAAGGGTCAGTCACCTTCTGTGTATTTTGCTG-3′, | (SEQ ID NO: 30) |
| and | |
| 5′-GCGGTCCCAAAAGGGTCAGTAAGTATTGGACAACTTGTTAGTCTC-3′ | (SEQ ID NO: 31) |
| and | |
| 5′-GCGGTCCCAAAAGGGTCAGTCGCCTTTCCAGTTGTATAATTT-3′. | (SEQ ID NO: 32) |

11. The method of claim 10, wherein said one or more pairs of nucleic acid primers is five pairs of nucleic acid primers.

12. The method of claim 10, wherein said one or more pairs of nucleic acid primers is ten pairs of nucleic acid primers.

13. The method of claim 10, wherein said one or more pairs of nucleic acid primers is fifteen pairs of nucleic acid primers.

14. The method of claim 13, wherein said primer sets are added in the following ratios determined as the mass of primers for exon 12 and 21 (SEQ ID NO: 9, 10, 13 and 14) to the mass of each other primer sets, the ratio being about 2 for exons 4 and i19 (SEQ ID NOs; 3-6), about 3.2 for exons 19, 7 and i5 (SEQ ID NOs; 7, 8, 15, 16, and 29-32), about 4 for exons 3 and 14 (SEQ ID NOs; 11, 12, 19, 20), about 4.8 for exons 16, 20, 13 and 10 (SEQ ID NOs; 17, 18, 23 and 28), and about 8 for exon 9 (SEQ ID NOs; 22 and 21).

15. The method of claim 10, wherein said step of amplifying is the polymerase chain reaction.

16. The method of claim 10, wherein said step of identifying the presence or absence of said one or more predetermined sequences is preformed using a solid phase array of nucleic acid probes complementary to said nucleic acid sequences that are correlated with cystic fibrosis.

17. A method of determining whether a subject has a genotype containing one or more nucleotide sequences correlated with cystic fibrosis, comprising:
obtaining a sample of nucleic acid from the subject;
contacting said sample with reagents suitable for nucleic acid amplification including one or more pairs of nucleic acid primers flanking one or more predetermined nucleic acid sequences that are correlated with cystic fibrosis,
amplifying said predetermined nucleic acid sequence(s), if present, to provide an amplified sample; and
identifying the presence of said one or more nucleic acid sequences correlated with cystic fibrosis nucleic, whereby the presence of one or more nucleic acid sequences correlated with cystic fibrosis in the genotype of the subject is determined;
wherein said pairs of nucleic acid primers are selected from the group consisting of:

| | |
|---|---|
| 5′-GCGGTCCCAAAAGGGTCAGTTGTAGGAAGTCACCAAAG-3′ | (SEQ ID NO: 3) |
| and | |
| 5′-GCGGTCCCAAAAGGGTCAGTCGATACAGAATATATGTGCC-3′, | (SEQ ID NO: 4) |
| 5′-GCGGTCCCAAAAGGGTCAGTGAATCATTCAGTGGGTATAAGCAG-3′ | (SEQ ID NO: 5) |
| and | |
| 5′-GCGGTCCCAAAAGGGTCAGTCTTCAATGCACCTCCTCCC-3′, | (SEQ ID NO: 6) |
| 5′-GCGGTCCCAAAAGGGTCAGTAGATACTTCAATAGCTCAGCC-3′ | (SEQ ID NO: 7) |
| and | |
| 5′-GCGGTCCCAAAAGGGTCAGTGGTACATTACCTGTATTTTGTTT-3′, | (SEQ ID NO: 8) |
| 5′-GCGGTCCCAAAAGGGTCAGTGTGAATCGATGTGGTGACCA-3′ | (SEQ ID NO: 9) |
| and | |
| 5′-GCGGTCCCAAAAGGGTCAGTCTGGTTTAGCATGAGGCGGT-3′, | (SEQ ID NO: 10) |
| 5′-GCGGTCCCAAAAGGGTCAGTTTGGTTGTGCTGTGGCTCCT-3′ | (SEQ ID NO: 11) |
| and | |
| 5′-GCGGTCCCAAAAGGGTCAGTACAATACATACAAACATAGTGG-3′, | (SEQ ID NO: 12) |
| 5′-GCGGTCCCAAAAGGGTCAGTGAAAGTATTTATTTTTTCTGGAAC-3′ | (SEQ ID NO: 13) |
| and | |
| 5′-GCGGTCCCAAAAGGGTCAGTGTGTGTAGAATGATGTCAGCTAT-3′, | (SEQ ID NO: 14) |
| 5′-GCGGTCCCAAAAGGGTCAGTCAGATTGAGCATACTAAAAGTG-3′ | (SEQ ID NO: 15) |
| and | |
| 5′-GCGGTCCCAAAAGGGTCAGTTACATGAATGACATTTACAGCA-3′, | (SEQ ID NO: 16) |
| 5′-GCGGTCCCAAAAGGGTCAGTAAGAACTGGATCAGGGAAGA-3′ | (SEQ ID NO: 17) |
| and | |
| 5′-GCGGTCCCAAAAGGGTCAGTTCCTTTTGCTCACCTGTGGT-3′, | (SEQ ID NO: 18) |
| 5′-GCGGTCCCAAAAGGGTCAGTGGTCCCACTTTTTATTCTTTTGC-3′ | (SEQ ID NO: 19) |
| and | |
| 5′-GCGGTCCCAAAAGGGTCAGTTGGTTTCTTAGTGTTTGGAGTTG-3′, | (SEQ ID NO: 20) |
| 5′-GCGGTCCCAAAAGGGTCAGTTGGATCATGGGCCATGTGC-3′ | (SEQ ID NO: 21) |
| and | |
| 5′-GCGGTCCCAAAAGGGTCAGTACTACCTTGCCTGCTCCAGTGG-3′, | (SEQ ID NO: 22) |
| 5′-GCGGTCCCAAAAGGGTCAGTAGGTAGCAGCTATTTTTATGG-3′ | (SEQ ID NO: 23) |
| and | |
| 5′-GCGGTCCCAAAAGGGTCAGTTAAGGGAGTCTTTTGCACAA-3′, | (SEQ ID NO: 24) |
| 5′-GCGGTCCCAAAAGGGTCAGTGCAATTTTGGATGACCTTC-3′ | (SEQ ID NO: 25) |
| and | |
| 5′-GCGGTCCCAAAAGGGTCAGTTAGACAGGACTTCAACCCTC-3′, | (SEQ ID NO: 26) |
| 5′-GCGGTCCCAAAAGGGTCAGTGGTGATTATGGGAGAACTGG-3′ | (SEQ ID NO: 27) |
| and | |
| 5′-GCGGTCCCAAAAGGGTCAGTATGCTTTGATGACGCTTC-3′, | (SEQ ID NO: 28) |
| 5′-GCGGTCCCAAAAGGGTCAGTTTCATTGAAAAGCCCGAC-3′ | (SEQ ID NO: 29) |
| and | |
| 5′-GCGGTCCCAAAAGGGTCAGTCACCTTCTGTGTATTTTGCTG-3′, | (SEQ ID NO: 30) |
| and | |
| 5′-GCGGTCCCAAAAGGGTCAGTAAGTATTGGACAACTTGTTAGTCTC-3′ | (SEQ ID NO: 31) |
| and | |
| 5′-GCGGTCCCAAAAGGGTCAGTCGCCTTTCCAGTTGTATAATTT-3′. | (SEQ ID NO: 32) |

18. The method of claim 17, wherein said one or more pairs of nucleic acid primers is five pairs of nucleic acid primers.

19. The method of claim 17, wherein said one or more pairs of nucleic acid primers is ten pairs of nucleic acid primers.

20. The method of claim 17, wherein said one or more pairs of nucleic acid primers is fifteen pairs of nucleic acid primers.

21. The method of claim 20, wherein said primer sets are added in the following ratios determined as the moles of primers for exon 12 and 21 (SEQ ID NO: 9, 10, 13 and 14) to the moles of each other primer sets, the ratio being about 2 for exons 4 and i19 (SEQ ID NOs; 3-6), about 3.2 for exons 19, 7, 11 and i5 (SEQ ID NOs; 7, 8, 15, 16, and 29-32), about 4 for exons 3 and 14 (SEQ ID NOs; 11, 12, 19, 20), about 4.8 for exons 16, 20, 13 and 10 (SEQ ID NOs; 17, 18, 23 and 28), and about 8 for exon 9 (SEQ ID NOs; 22 and 21).

22. The method of claim 17, wherein said step of amplifying is the polymerase chain reaction.

23. The method of claim 17, wherein said step of identifying the presence of said one or more sequences correlated with cystic fibrosis is preformed using a solid phase array of nucleic acid probes complementary to said nucleic acid sequences correlated with cystic fibrosis.

24. A kit for amplifying sequences of the cystic fibrosis CTFR gene comprising one or more pairs of nucleic acid primers selected from the group consisting of:

| | |
|---|---|
| 5′-GCGGTCCCAAAAGGGTCAGTTGTAGGAAGTCACCAAAG-3′ | (SEQ ID NO: 3) |
| and | |
| 5′-GCGGTCCCAAAAGGGTCAGTCGATACAGAATATATGTGCC-3′, | (SEQ ID NO: 4) |
| 5′-GCGGTCCCAAAAGGGTCAGTGAATCATTCAGTGGGTATAAGCAG-3′ | (SEQ ID NO: 5) |
| and | |
| 5′-GCGGTCCCAAAAGGGTCAGTCTTCAATGCACCTCCTCCC-3′, | (SEQ ID NO: 6) |
| 5′-GCGGTCCCAAAAGGGTCAGTAGATACTTCAATAGCTCAGCC-3′ | (SEQ ID NO: 7) |
| and | |
| 5′-GCGGTCCCAAAAGGGTCAGTGGTACATTACCTGTATTTTGTTT-3′, | (SEQ ID NO: 8) |
| 5′-GCGGTCCCAAAAGGGTCAGTGTGAATCGATGTGGTGACCA-3′ | (SEQ ID NO: 9) |
| and | |
| 5′-GCGGTCCCAAAAGGGTCAGTCTGGTTTAGCATGAGGCGGT-3′, | (SEQ ID NO: 10) |
| 5′-GCGGTCCCAAAAGGGTCAGTTTGGTTGTGCTGTGGCTCCT-3′ | (SEQ ID NO: 11) |
| and | |
| 5′-GCGGTCCCAAAAGGGTCAGTACAATACATACAAACATAGTGG-3′, | (SEQ ID NO: 12) |
| 5′-GCGGTCCCAAAAGGGTCAGTGAAAGTATTTATTTTTTCTGGAAC-3′ | (SEQ ID NO: 13) |
| and | |
| 5′-GCGGTCCCAAAAGGGTCAGTGTGTGTAGAATGATGTCAGCTAT-3′, | (SEQ ID NO: 14) |
| 5′-GCGGTCCCAAAAGGGTCAGTCAGATTGAGCATACTAAAAGTG-3′ | (SEQ ID NO: 15) |
| and | |
| 5′-GCGGTCCCAAAAGGGTCAGTTACATGAATGACATTTACAGCA-3′, | (SEQ ID NO: 16) |
| 5′-GCGGTCCCAAAAGGGTCAGTAAGAACTGGATCAGGGAAGA-3′ | (SEQ ID NO: 17) |
| and | |
| 5′-GCGGTCCCAAAAGGGTCAGTTCCTTTTGCTCACCTGTGGT-3′, | (SEQ ID NO: 18) |
| 5′-GCGGTCCCAAAAGGGTCAGTGGTCCCACTTTTTATTCTTTTGC-3′ | (SEQ ID NO: 19) |
| and | |
| 5′-GCGGTCCCAAAAGGGTCAGTTGGTTTCTTAGTGTTTGGAGTTG-3′, | (SEQ ID NO: 20) |
| 5′-GCGGTCCCAAAAGGGTCAGTTGGATCATGGGCCATGTGC-3′ | (SEQ ID NO: 21) |
| and | |
| 5′-GCGGTCCCAAAAGGGTCAGTACTACCTTGCCTGCTCCAGTGG-3′, | (SEQ ID NO: 22) |
| 5′-GCGGTCCCAAAAGGGTCAGTAGGTAGCAGCTATTTTTATGG-3′ | (SEQ ID NO: 23) |
| and | |
| 5′-GCGGTCCCAAAAGGGTCAGTTAAGGGAGTCTTTTGCACAA-3′, | (SEQ ID NO: 24) |
| 5′-GCGGTCCCAAAAGGGTCAGTGCAATTTTGGATGACCTTC-3′ | (SEQ ID NO: 25) |
| and | |
| 5′-GCGGTCCCAAAAGGGTCAGTTAGACAGGACTTCAACCCTC-3′, | (SEQ ID NO: 26) |
| 5′-GCGGTCCCAAAAGGGTCAGTGGTGATTATGGGAGAACTGG-3′ | (SEQ ID NO: 27) |
| and | |
| 5′-GCGGTCCCAAAAGGGTCAGTATGCTTTGATGACGCTTC-3′, | (SEQ ID NO: 28) |
| 5′-GCGGTCCCAAAAGGGTCAGTTTCATTGAAAAGCCCGAC-3′ | (SEQ ID NO: 29) |
| and | |
| 5′-GCGGTCCCAAAAGGGTCAGTCACCTTCTGTGTATTTTGCTG-3′, | (SEQ ID NO: 30) |
| and | |
| 5′-GCGGTCCCAAAAGGGTCAGTAAGTATTGGACAACTTGTTAGTCTC-3′ | (SEQ ID NO: 31) |
| and | |
| 5′-GCGGTCCCAAAAGGGTCAGTCGCCTTTCCAGTTGTATAATTT-3′, | (SEQ ID NO: 32) |

in an amount sufficient to perform a polymerase chain reaction amplification of a nucleic acid sample.

25. The kit of claim 24, wherein said one or more pairs of nucleic acid primers is five pairs of nucleic acid primers.

26. The kit of claim 24, wherein said one or more pairs of nucleic acid primers is ten pairs of nucleic acid primers.

27. The kit of claim 24, wherein said one or more pairs of nucleic acid primers is fifteen pairs of nucleic acid primers.


SEQUENCE LISTING (36 sequences)

SEQ ID NO: 1 - DNA, 13 bp, Artificial Sequence
  Description of Artificial Sequence Synthetic oligonucleotide
gcggtcccaaaag

SEQ ID NO: 2 - DNA, 13 bp, Artificial Sequence
  Description of Artificial Sequence Synthetic oligonucleotide
cttttgggaccgc

SEQ ID NO: 3 - DNA, 38 bp, Artificial Sequence
  Description of Artificial Sequence Synthetic oligonucleotide
gcggtcccaaaagggtcagttgtaggaagtcaccaaag

SEQ ID NO: 4 - DNA, 40 bp, Artificial Sequence
  Description of Artificial Sequence Synthetic oligonucleotide
gcggtcccaaaagggtcagtcgatacagaatatatgtgcc

SEQ ID NO: 5 - DNA, 44 bp, Artificial Sequence
  Description of Artificial Sequence Synthetic oligonucleotide
gcggtcccaaaagggtcagtgaatcattcagtgggtataagcag

SEQ ID NO: 6 - DNA, 39 bp, Artificial Sequence
  Description of Artificial Sequence Synthetic oligonucleotide
gcggtcccaaaagggtcagtcttcaatgcacctcctccc

SEQ ID NO: 7 - DNA, 41 bp, Artificial Sequence
  Description of Artificial Sequence Synthetic oligonucleotide
gcggtcccaaaagggtcagtagatacttcaatagctcagcc

SEQ ID NO: 8 - DNA, 43 bp, Artificial Sequence
  Description of Artificial Sequence Synthetic oligonucleotide
gcggtcccaaaagggtcagtggtacattacctgtattttgttt

SEQ ID NO: 9 - DNA, 40 bp, Artificial Sequence
  Description of Artificial Sequence Synthetic oligonucleotide
gcggtcccaaaagggtcagtgtgaatcgatgtggtgacca

SEQ ID NO: 10 - DNA, 40 bp, Artificial Sequence
  Description of Artificial Sequence Synthetic oligonucleotide
gcggtcccaaaagggtcagtctggtttagcatgaggcggt

SEQ ID NO: 11 - DNA, 40 bp, Artificial Sequence
  Description of Artificial Sequence Synthetic oligonucleotide
gcggtcccaaaagggtcagtttggttgtgctgtggctcct

SEQ ID NO: 12 - DNA, 42 bp, Artificial Sequence
  Description of Artificial Sequence Synthetic oligonucleotide
gcggtcccaaaagggtcagtacaatacatacaaacatagtgg

SEQ ID NO: 13 - DNA, 44 bp, Artificial Sequence
  Description of Artificial Sequence Synthetic oligonucleotide
gcggtcccaaaagggtcagtgaaagtatttattttttctggaac

SEQ ID NO: 14 - DNA, 43 bp, Artificial Sequence
  Description of Artificial Sequence Synthetic oligonucleotide
gcggtcccaaaagggtcagtgtgtgtagaatgatgtcagctat

SEQ ID NO: 15 - DNA, 42 bp, Artificial Sequence
  Description of Artificial Sequence Synthetic oligonucleotide
gcggtcccaaaagggtcagtcagattgagcatactaaaagtg

SEQ ID NO: 16 - DNA, 42 bp, Artificial Sequence
  Description of Artificial Sequence Synthetic oligonucleotide
gcggtcccaaaagggtcagttacatgaatgacatttacagca

SEQ ID NO: 17 - DNA, 40 bp, Artificial Sequence
  Description of Artificial Sequence Synthetic oligonucleotide
gcggtcccaaaagggtcagtaagaactggatcagggaaga

SEQ ID NO: 18 - DNA, 40 bp, Artificial Sequence
  Description of Artificial Sequence Synthetic oligonucleotide
gcggtcccaaaagggtcagttccttttgctcacctgtggt

SEQ ID NO: 19 - DNA, 43 bp, Artificial Sequence
  Description of Artificial Sequence Synthetic oligonucleotide
gcggtcccaaaagggtcagtggtcccactttttattcttttgc

SEQ ID NO: 20 - DNA, 43 bp, Artificial Sequence
  Description of Artificial Sequence Synthetic oligonucleotide
gcggtcccaaaagggtcagttggtttcttagtgtttggagttg

SEQ ID NO: 21 - DNA, 39 bp, Artificial Sequence
  Description of Artificial Sequence Synthetic oligonucleotide
gcggtcccaaaagggtcagttggatcatgggccatgtgc

SEQ ID NO: 22 - DNA, 42 bp, Artificial Sequence
  Description of Artificial Sequence Synthetic oligonucleotide
gcggtcccaaaagggtcagtactaccttgcctgctccagtgg

SEQ ID NO: 23 - DNA, 41 bp, Artificial Sequence
  Description of Artificial Sequence Synthetic oligonucleotide
gcggtcccaaaagggtcagtaggtagcagctatttttatgg

SEQ ID NO: 24 - DNA, 40 bp, Artificial Sequence
  Description of Artificial Sequence Synthetic oligonucleotide
gcggtcccaaaagggtcagttaagggagtcttttgcacaa

SEQ ID NO: 25 - DNA, 39 bp, Artificial Sequence
  Description of Artificial Sequence Synthetic oligonucleotide
gcggtcccaaaagggtcagtgcaattttggatgaccttc

SEQ ID NO: 26 - DNA, 40 bp, Artificial Sequence
  Description of Artificial Sequence Synthetic oligonucleotide
gcggtcccaaaagggtcagttagacaggacttcaaccctc

SEQ ID NO: 27 - DNA, 40 bp, Artificial Sequence
  Description of Artificial Sequence Synthetic oligonucleotide
gcggtcccaaaagggtcagtggtgattatgggagaactgg

SEQ ID NO: 28 - DNA, 38 bp, Artificial Sequence
  Description of Artificial Sequence Synthetic oligonucleotide
gcggtcccaaaagggtcagtatgctttgatgacgcttc

SEQ ID NO: 29 - DNA, 38 bp, Artificial Sequence
  Description of Artificial Sequence Synthetic oligonucleotide
gcggtcccaaaagggtcagtttcattgaaaagcccgac

SEQ ID NO: 30 - DNA, 41 bp, Artificial Sequence
  Description of Artificial Sequence Synthetic oligonucleotide
gcggtcccaaaagggtcagtcaccttctgtgtattttgctg

SEQ ID NO: 31 - DNA, 45 bp, Artificial Sequence
  Description of Artificial Sequence Synthetic oligonucleotide
gcggtcccaaaagggtcagtaagtattggacaacttgttagtctc

SEQ ID NO: 32 - DNA, 42 bp, Artificial Sequence
  Description of Artificial Sequence Synthetic oligonucleotide
gcggtcccaaaagggtcagtcgcctttccagttgtataattt

SEQ ID NO: 33 - DNA, 21 bp, Artificial Sequence
  Description of Artificial Sequence Synthetic oligonucleotide
ccatttttggccttcatcaca

SEQ ID NO: 34 - DNA, 32 bp, Artificial Sequence
  Description of Artificial Sequence Synthetic oligonucleotide
gatcgatctgtctcctggacagaaacaaaaaa

SEQ ID NO: 35 - DNA, 37 bp, Artificial Sequence
  Description of Artificial Sequence Synthetic oligonucleotide
gactgatcgatcgttattgaatcccaagacacaccat

SEQ ID NO: 36 - DNA, 43 bp, Artificial Sequence
  Description of Artificial Sequence Synthetic oligonucleotide
gactgatcgatcgatccctcttaccatatttgacttcatccag